(19) United States
(12) Patent Application Publication
(10) Pub. No.: US 2009/0093679 A1
(43) Pub. Date: Apr. 9, 2009

(54) COUPLING STRUCTURE FOR ENDOSCOPE FLEXIBLE TUBE AND ANNULAR COUPLING MEMBER

(75) Inventors: Naoki SUIGETSU, Kokubunji-shi (JP); Hi deya KITAGAWA, Hachioji-shi (JP); Yoshiaki ITO, Fuchu-shi (JP); Takeshi KIDA, Kunitachi-shi (JP)

Correspondence Address: FRISHAUF, HOLTZ, GOODMAN & CHICK, P.C., 220 FIFTH AVENUE - 16TH FLOOR, NEW YORK, NY, 10001-7708, US

(73) Assignee: OLYMPUS CORPORATION, Tokyo (JP)

(21) Appl. No.: 12/330,797

(22) Filed: Dec. 9, 2008

Related U.S. Application Data
(63) Continuation of application No. PCT/JP2008/056489, filed on Apr. 1, 2008.

(30) Foreign Application Priority Data
Apr. 11, 2007 (JP) 2007-104118

(51) Int. Cl. A61B 1/00

(52) U.S. Cl. 600/139

(57) ABSTRACT

An insertion section of an endoscope has a flexible tube portion and a bending portion whose proximal end portion is coupled with a distal end of the flexible tube portion, a plurality of node rings are aligned in a bending tube serving as a base body portion of the bending portion, opening portions as through openings are provided in a node ring, protruding portions each having substantially the same shape as the opening portion are provided on an outer peripheral surface of a joint portion of the flexible tube portion that is inserted into the node ring, and a coupling structure according to the present invention couples the bending portion with the flexible tube portion by inserting the joint portion into the node ring and fitting the protruding portions into the opening portions.

### CROSS-REFERENCE TO RELATED APPLICATIONS

This is a Continuation Application of PCT Application No. PCT/JP2008/056489, filed Apr. 1, 2008, which was published under PCT Article 21(2) in Japanese.

This application is based upon and claims the benefit of priority from prior Japanese Patent Application No. 2007-104118, filed Apr. 11, 2007, the entire contents of which are incorporated herein by reference.

### BACKGROUND OF THE INVENTION

1. Field of the Invention

The present invention relates to a coupling structure for an endoscope flexible tube and an annular coupling member in an endoscope insertion section that is inserted into a body cavity.

2. Description of the Related Art

In general, an insertion section that is inserted into a body cavity and an operating section that is coupled with a proximal end portion of the insertion section to operate the insertion section are arranged in a soft endoscope. In the insertion section are provided an elongated flexible tube portion, a bending portion that is coupled with a distal end of this flexible tube portion and can freely bend, and a distal end hard portion that is arranged at the most distal end portion of the insertion section. A proximal end portion of the flexible tube portion is coupled with the operating section on an operator's hand side.

A plurality of node rings are aligned in the bending portion along an insertion (longitudinal axis) direction of the insertion section. The node rings that are adjacent to each other (placed at lengthwise positions along the insertion direction) are coupled by each spindle portion such as a rivet to allow their swiveling motion. In this bending portion, the node ring arranged at a position closest to the operating section is coupled with the distal end of the flexible tube portion. Further, distal end portions of four operation wires that bend the bending portion in, e.g., four directions of up, down, left, and right are fixed on the distal end side of the bending portion. Proximal end portions of these bending operation wires are extended to the operating section through the inside of the flexible tube portion.

Furthermore, each of Patent Document 1 and Patent Document 2 discloses a connection method (a coupling structure) for an endoscope flexible tube and an annular coupling member in an insertion section of a conventional endoscope.

Patent Document 1 discloses a flexible tube having a three-layer structure in which a metal net-like tube is disposed on a resin tube and a resin layer serving as an envelope is formed on the net-like tube. A coupling structure in this flexible tube enables removal of the resin layer at an end portion of the flexible tube to expose the net-like tube. Then, a solder is set out on an outer periphery of the exposed net-like tube, and the setout solder portion is cut at the midpoint thereof in a direction orthogonal to an axial line of the net-like tube. Subsequently, the resin tube placed at the end portion of the cut flexible tube is removed, and an outer diameter of the end portion including the solder portion is finished to become substantially equal to an outer diameter of the resin layer at the end portion. Moreover, one end of a coupling member is inserted into and fixed to a portion from which the resin tube is removed.

Additionally, according to a connection method disclosed in Patent Document 2, an annular coupling member having a large-diameter portion associated with an outer peripheral diameter of a net-like tube in a flexible tube, a small-diameter portion continuous with the large-diameter portion, and a radial through-hole formed in the large-diameter portion is prepared, and a resin layer at an end portion of the flexible tube is removed to expose the net-like tube. Then, a solder is set out on an outer periphery of the exposed net-like tube, and the setout solder portion is cut at the midpoint thereof in a direction orthogonal to an axial line of the net-like tube. Subsequently, an outer diameter at the end portion of the flexible tube including the solder portion is polished to become substantially equal to an outer peripheral diameter of the net-like tube. Further, the large-diameter portion of the annular coupling member is fitted on the outer periphery of the exposed end portion of the net-like tube at the end portion of the flexible tube, and the solder is flowed via the radial through-hole to couple the net-like tube with the annular coupling member.

### Patent Document 1: JP-A 2003-144384 (KOKAI)

### Patent Document 2: JP-A 2003-164421 (KOKAI)

### BRIEF SUMMARY OF THE INVENTION

In the coupling methods disclosed in Patent Document 1 and Patent Document 2, for example, removal of the resin layer, the process of finishing the outer diameter of the end portion including the solder portion to become substantially equal to the outer diameter of the resin layer at the end portion of the flexible tube, or the process of polishing the outer diameter at the end portion of the flexible tube to become substantially equal to the outer peripheral diameter of the net-like tube is complicated and not easy. Furthermore, in the coupling method based on insertion and fixation or soldering, coupling strength is insufficient, and a hard portion that cannot sufficiently bend may possibly become long.

Therefore, the present invention provides a coupling structure for an endoscope flexible tube and an annular coupling member that can simplify a process of coupling a bending portion with a flexible tube portion, easily realize coupling, and provide high coupling strength and a short hard portion.

According to one aspect of the present invention, there is provided a coupling structure for an endoscope flexible tube and an annular coupling member, comprising: a joint portion that is arranged at a distal end portion of a flexible tube portion for an endoscope and has a substantially annular shape; an annular coupling member that is provided to a bending portion arranged on a distal end side of the flexible tube portion and coupled with the joint portion; a coupling opening portion that is provided in the annular coupling member and used to couple the joint portion with the annular coupling member; and a protruding portion that is formed on an outer peripheral surface of the joint portion and configured to be fitted into the opening portion, wherein, when the protruding portion is fitted into the opening portion at the time of coupling that the joint portion is inserted into the annular coupling member to couple the flexible tube portion with the bending portion, the opening portion and the protruding portion form retaining means for preventing the joint portion coming off the annular coupling member.

### BRIEF DESCRIPTION OF THE SEVERAL VIEWS OF THE DRAWING

FIG. 1 is a schematic view showing an endoscope according to a first embodiment;

FIG. 2 is a view showing an example of a shape of a flexible tube;

FIG. 3 is a schematic view showing an internal structure of a distal end hard portion;

FIG. 4 is a cross-sectional view of a bending portion of the endoscope taken along line A-A depicted in FIG. 1;

FIG. 5 is a view showing an alignment state of node rings in the bending portion;

FIG. 6 is a perspective view showing a structure of a node ring that is arranged on a side closest to the distal end hard portion;

FIG. 7 is a perspective view showing a structure of a node ring that is arranged on a side closest to the flexible tube portion;

FIG. 8 is a perspective view when the bending portion is coupled with the flexible tube portion;

FIG. 9 is a perspective view showing the bending portion and the flexible tube portion coupled with each other;

FIG. 10 is a cross-sectional view of the bending portion and the flexible tube portion coupled with each other taken along line C-C depicted in FIG. 9;

FIG. 11 is a cross-sectional view of the bending portion and the flexible tube portion coupled with each other taken along line D-D depicted in FIGS. 9 and 10;

FIG. 12 is a cross-sectional view of a wire guide portion of the node ring taken along line B-B depicted in FIG. 6;

FIG. 13 is a schematic vertical sectional view partially showing a fracture section of a situation where the bending portion and the flexible tube portion coupled with each other are maintained in a non-bent state;

FIG. 14 is a perspective view when a bending tube is coupled with a flexible tube portion in a second embodiment;

FIG. 15 is a cross-sectional view when the bending tube is coupled with the flexible tube portion taken along line E-E depicted in FIG. 14;

FIG. 16A is a schematic perspective view of a joint portion including slits in a first modification;

FIG. 16B is a top view of the joint portion in the first modification;

FIG. 16C is a front view of the joint portion in the first modification;

FIG. 17A is a schematic perspective view of a joint portion including slits in a second modification;

FIG. 17B is a top view of the joint portion in the second modification;

FIG. 17C is a front view of the joint portion in the second modification;

FIG. 17D is a cross-sectional view of the joint portion taken along line F-F depicted in FIGS. 17A and 17B;

FIG. 18A is a schematic perspective view of a joint portion including slits in a third modification;

FIG. 18B is a top view of the joint portion in the third modification;

FIG. 18C is a front view of the joint portion in the third modification;

FIG. 19 is a schematic perspective view of a joint portion including slits in a fourth modification;

FIG. 20 is a schematic perspective view of a joint portion including slits in a fifth modification;

FIG. 21 is a schematic perspective view of a joint portion including slits and a node ring in a sixth modification;

FIG. 22 is a perspective view when a bending portion is coupled with a flexible tube portion in a third modification;

FIG. 23 is a perspective view showing the bending portion and the flexible tube portion coupled with each other;

FIG. 24 is a cross-sectional view showing the bending portion and the flexible tube portion coupled with each other taken along line G-G depicted in FIG. 23;

FIG. 25 is a cross-sectional view of a where a joint portion having slits but no anti-slip portion formed thereto is inserted into a node ring, protruding portions are fitted into opening portions, and the bending portion is coupled with the flexible tube portion taken along line G-G depicted in FIG. 23;

FIG. 26 is a cross-sectional view when a force is applied to the bending portion and the flexible tube portion coupled with each other in a bending direction taken along line G-G depicted in FIG. 23;

FIG. 27 is a cross-sectional view showing the bending portion and the flexible tube portion coupled with each other taken along line C-G depicted in FIG. 23 when a force is applied to the bending portion and the flexible tube portion coupled with each other in the bending direction in the third embodiment;

FIG. 28 is a schematic perspective view of a joint portion including an anti-slip portion in a first modification;

FIG. 29A is a schematic perspective view of a joint portion including a step portion as an anti-slip portion in a second modification;

FIG. 29B is a cross-sectional view of a state where the joint portion in the second modification is coupled with a node ring taken along line G-G depicted in FIG. 23;

FIG. 30A is a schematic perspective view of a node ring including an anti-slip portion and a joint portion in a third modification;

FIG. 30B is a cross-sectional view of a state where the joint portion is coupled with the node ring taken along line H-H depicted in FIG. 30A;

FIG. 31A is a schematic perspective view of a joint portion including an anti-slip portion and a node ring in a fourth modification;

FIG. 31B is a cross-sectional view of a state where the joint portion is coupled with the node ring taken along line I-I depicted in FIG. 31A;

FIG. 32A is a schematic perspective view of a node ring including an anti-slip portion and a joint portion in a fifth modification;

FIG. 32B is a cross-sectional view of a state where the joint portion is coupled with the node ring taken along line J-J depicted in FIG. 32A;

FIG. 33 is a perspective view when a node ring is coupled with a joint portion in a fourth embodiment;

FIG. 34A is a perspective view of a node ring and a joint portion coupled with each other;

FIG. 34B is a cross-sectional view when a force is applied to a bending portion and a flexible tube portion coupled with each other in a bending direction taken along line K-K depicted in FIG. 33;

FIG. 35A is a schematic perspective view of a joint portion in a first modification;

FIG. 35B is a cross-sectional view of a state where a slit insertion member is inserted in the joint portion and the node ring coupled with each other taken along line L-L depicted in FIG. 35A;

FIG. 36A is a cross-sectional view of a state where a slit insertion member is inserted into a joint portion and a node ring coupled with each other in a second modification taken along line L-L depicted in FIG. 35A;

FIG. 36B is a cross-sectional view of a state where the slit insertion member is inserted in the joint portion and the node ring coupled with each other taken along line L-L depicted in FIG. 35A;

FIG. 37A is a cross-sectional view of a state where a slit insertion member is inserted into a joint portion and a node ring coupled with each other in a third modification taken along line L-L depicted in FIG. 35A;

FIG. 37B is a side view of a state where the slit insertion member is inserted in the joint portion and the node ring coupled with each other;

FIG. 38A is a cross-sectional view of a state where a slit insertion member is inserted into a joint portion and a node ring coupled with each other in a fourth modification taken along line L-L depicted in FIG. 35A;

FIG. 38B is a side view of a state where the slit insertion member is inserted in the joint portion and the node ring coupled with each other;

FIG. 39 is a perspective view of a state where a slit insertion member is inserted into a joint portion and a node ring coupled with each other in a fifth modification;

FIG. 40 is a perspective view when a node ring is coupled with a joint portion in a fifth embodiment;

FIG. 41 is a cross-sectional view of the node ring and the joint portion coupled with each other taken along line M-M depicted in FIG. 40;

FIG. 42 is a cross-sectional view of the node ring and the joint portion coupled with each other taken along line N-N depicted in FIGS. 40 and 41;

FIG. 43 is a cross-sectional view when the joint portion is inserted into the node ring taken along line M-M depicted in FIG. 40;

FIG. 44 is a cross-sectional view of the node ring and the joint portion coupled with each other when a hot melt is inserted in a space portion taken along line M-M depicted in FIG. 40;

FIG. 45 is a cross-sectional view of the node ring and the joint portion coupled with each other when the hot melt is inserted in the space portion taken along line M-M depicted in FIG. 40;

FIG. 46 is a cross-sectional view of a node ring and a joint portion coupled with each other when screws as a first modification of a anti-bending members are inserted in a space portion taken along line M-M depicted in FIG. 40;

FIG. 47 is a cross-sectional view of a node ring and a joint portion coupled with each other when pins as a second modification of the anti-bending members are inserted in space portions taken along line M-M depicted in FIG. 40;

FIG. 48 is a side view of the node ring and the joint portion coupled with each other depicted in FIG. 47; and

FIG. 49 is a cross-sectional view showing a modification of the joint portion in the fifth embodiment taken along line M-M depicted in FIG. 40.

### DETAILED DESCRIPTION OF THE INVENTION

Embodiments according to the present invention will now be explained hereinafter in detail with reference to the drawings.

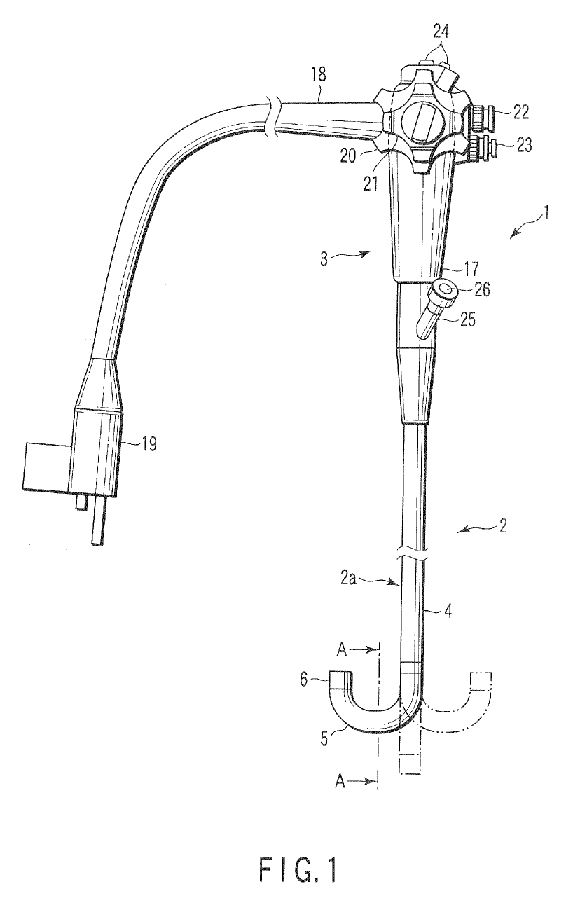

A first embodiment will be explained with reference to FIGS. 1 to 13.

As shown in FIG. 1, in an endoscope 1, an elongated insertion section 2 that is inserted into a body cavity of a patient and an operating section 3 that is coupled with a proximal end of the insertion section 2 placed on an operator's hand side to operate the insertion section 2 are provided.

To an insertion section main body portion 2a of the insertion section 2 are provided an elongated flexible tube portion (a corrugated tube portion) 4 having a proximal end portion coupled with the operating section 3, a bending portion 5 having a proximal end portion coupled with a distal end of this flexible tube portion 4, and a distal end hard portion 6 having a proximal end portion coupled with a distal end of this bending portion 5 are provided.

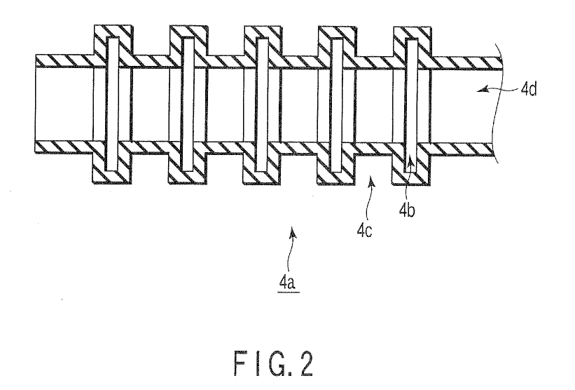

The flexible tube portion 4 has, e.g., a hollow shape formed of a resin. It is to be noted that the flexible tube portion 4 does not have to be restricted to this shape. An endoscope corrugated tube 4a formed of, e.g., a resin may be used for the flexible tube portion 4. The corrugated tube 4a is constituted of a hollow continuous body (a hollow body) 4d having a mountain-valley structure (a corrugated structure) formed of mountain portions 4b and valley portions 4c in a direction orthogonal to a longitudinal axis direction of the corrugated tube 4a as shown in FIG. 2, for example. That is, the hollow continuous body 4d is, e.g., a corrugated tube, and it may be used as the corrugated tube 4a itself.

The bending portion 5 can be bent into a bent state that the bending portion is bent as indicated by a solid line or a chain double-dashed line in FIG. 1 from a regular straight line state that the bending portion is straight as indicated by a dashed line in FIG. 1.

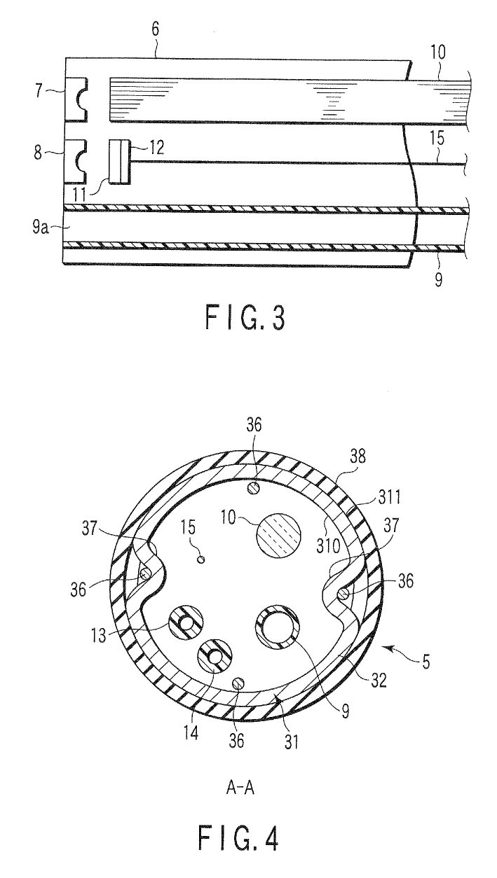

On a distal end surface of the distal end hard portion 6, an illumination lens 7 of an illumination optical system, an object lens 8 of an observation optical system, a distal end opening portion 9a of a surgical instrument insertion channel 9, a non-illustrated air supply/water supply nozzle, and suchlike are arranged as shown in FIG. 3. Furthermore, in the distal end hard portion 6, a distal end portion of a light guide fiber 10 is fixed behind the illumination lens 7. Moreover, an imaging element 11 such as a CCD, its connection circuit board 12, and suchlike are fixed behind the object lens 8. A cable 15 such as a signal line of the imaging element 11 is connected with the connection circuit board 12. It is to be noted that a distal end portion of a non-illustrated image guide fiber may be fixed in place of the imaging element 11 and the endoscope 1 may be a fiber scope without being restricted to an electronic scope. Additionally, a distal end portion of the surgical instrument insertion channel 9, distal end portions of an air supply tube 13 (see FIG. 4) and a water supply tube 14 (see FIG. 4) connected with the air supply/water supply nozzle, and suchlike are fixed to the distal end hard portion 6.

Further, the light guide fiber 10, the cable 15, the non-illustrated image guide fiber in case of the fiber scope, the surgical instrument insertion channel 9, the air supply tube 13, the water supply tube 14, and suchlike are extended to a proximal end portion side of the flexible tube portion 4 from the inside of the bending portion 5 through the inside of the flexible tube portion 4.

A grasping portion 17 grasped by an operator is arranged on the operating section 3. A proximal end portion of a universal cord 18 is coupled with this grasping portion 17. A connector portion 19 connected with a non-illustrated light source device or video processor is coupled with a distal end portion of this universal cord 18.

Furthermore, to the operating section 3 are provided a vertical (up and down) bending operation knob 20 that vertically bends the bending portion 5, a lateral (left and right) bending operation knob 21 that laterally bends the bending portion 5, a suction button 22, an air supply/water supply button 23, a various kinds of buttons 24 for endoscope imaging, and a surgical instrument insertion portion 25. A surgical instrument insertion opening 26 coupled with a proximal end portion of the surgical instrument insertion channel 9 arranged in the insertion section 2 is provided to the surgical instrument insertion portion 25. Further, a non-illustrated endoscope surgical instrument is inserted into the surgical instrument insertion channel 9 from the surgical instrument insertion opening 26 to be pushed in toward the distal end hard portion 6 side, and then protruded (projected) toward the outside from the distal end opening portion 9a of the surgical instrument insertion channel 9.

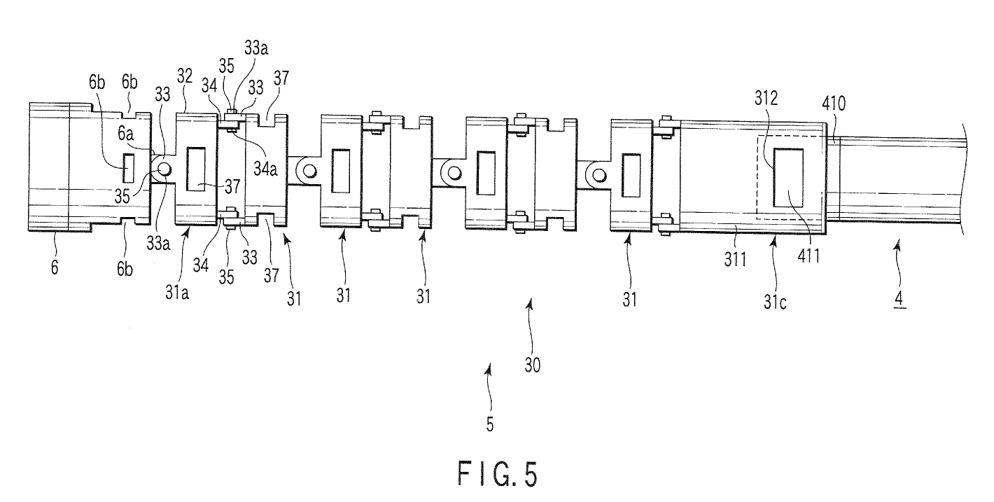

Then, a structure of the bending portion 5 will now be explained in detail. As shown in FIG. 5, a bending tube (an annular cylindrical member) 30 serving as a base body portion of the bending portion 5 is provided to the bending portion 5 arranged on the distal end side of the flexible tube portion 4. The bending tube 30 as an annular coupling member is coupled with a joint portion 410 of the flexible tube portion 4. The bending tube 30 has a plurality of node rings 31 aligned along an insertion (longitudinal axis) direction of the insertion section 2. The node ring 31 has a substantially cylindrical shape. The node rings 31 adjacent to each other (placed at lengthwise positions along the insertion direction of the endoscope 1) are coupled with each other through a spindle portion (e.g., a later-explained rivet 35) to allow their swiveling motion. The node ring 31 is formed of a hard material such as a metal. When the node rings 31 are coupled with each other in this manner, the bending tube 30 as the annular cylindrical member is formed. That is, the node ring 31 also functions as the annular cylindrical member. In the bending tube 30, the distal end hard portion 6 is coupled with a node ring 31 arranged at a position closest to the distal end hard portion 6 (its detail will be explained later). Furthermore, the joint portion 410 of the flexible tube portion 4 is coupled with a node ring 31c arranged at a position closest to the operating section 3 (its detail will be explained later).

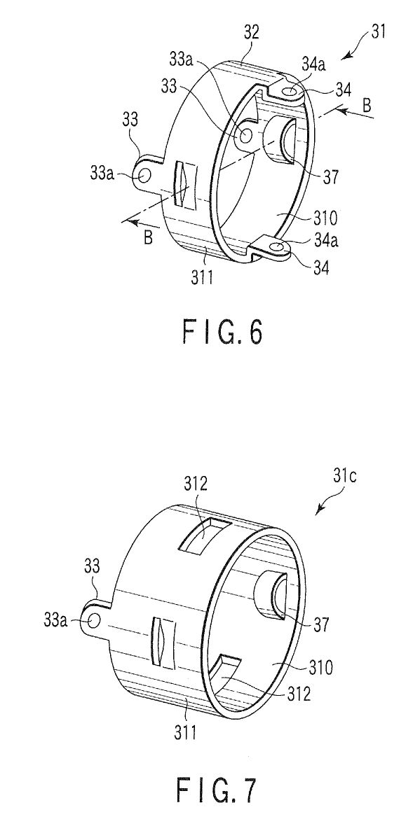

A structure of the node ring 31 will now be explained. As shown in FIG. 6, each node ring 31 has a node ring main body 32 having a substantially cylindrical shape. The node ring main body 32 is formed of, e.g., a metal thin plate pressed product or a cast. Two protruding (projecting) pieces (front hinge pedestals) 33 obtained by partially protruding an outer peripheral surface 311 of the node ring main body 32 toward the front side are arranged at a distal end portion of the node ring main body 32. The two protruding pieces 33 are arranged to be apart from each other at substantially 180° in a circumferential direction. Moreover, two protruding pieces (back hinge pedestals) 34 obtained by partially protruding the outer peripheral surface 311 of the node ring main body 32 toward the back side with a step substantially corresponding to a board thickness of the protruding piece 33 are arranged at a rear end portion of the node ring main body 32. The two protruding pieces 34 are arranged to be apart from each other at substantially 180° in the circumferential direction. The two protruding pieces 33 and the two protruding pieces 34 are arranged at positions apart from each other at substantially 90° in the circumferential direction. A hole 33a is formed in each protruding piece 33, and a hole 34a is formed in each protruding piece 34.

Coupling the node rings 31 with each other will now be explained. When the protruding pieces 34 of the front node ring 31 overlap the protruding pieces 33 of the rear node ring 31, the rivets 35 are inserted into the holes 33a and 34a. As a result, the front node ring 31 is coupled with the rear node ring 31 through the rivets 35, and they are supported about their axes to allow their swiveling motion on the rivets 35. A spindle portion using each rivet 35 as a swiveling spindle is formed between the protruding piece 34 and the protruding piece 33.

Coupling the node ring 31a with the distal end hard portion 6 will now be explained. As shown in FIG. 5, two protruding pieces 6a protruding toward the rear side are provided at the rear end portion of the distal end hard portion 6. The front two protruding pieces 33 of the node ring 31a are coupled with the two protruding pieces 6a through the rivets 35 as explained above so that they are supported about their axes to allow their swiveling motion on the rivets 35.

In the bending portion 5 according to this embodiment, the rivets 35 that couple the plurality of node rings 31 with each other and serve as the swiveling spindles are alternately arranged at substantially 90 degrees between the respective front and rear node rings 31. As a result, the entire bending portion 5 is configured to bend in four directions, i.e., up, down, left, and right.

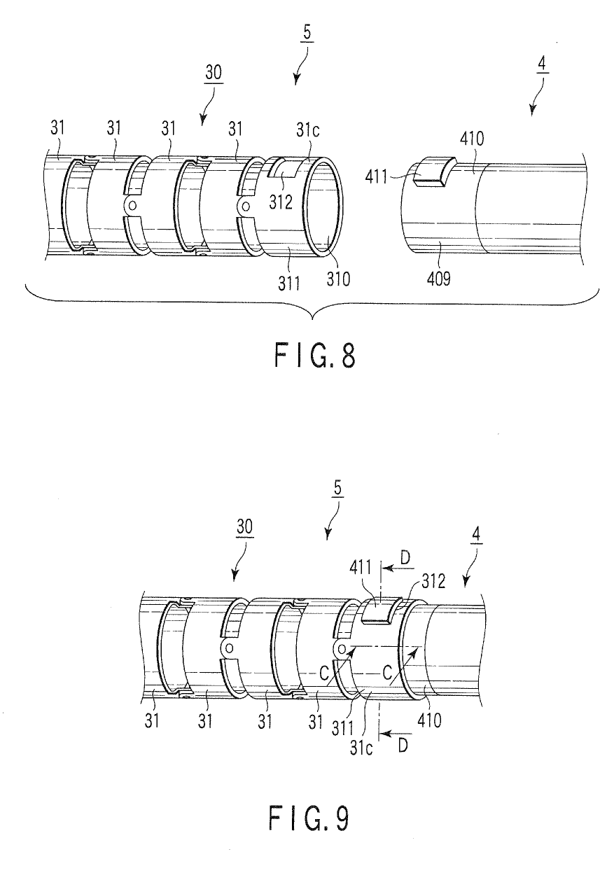

Coupling the bending portion 5 with the flexible tube portion 4 will now be briefly explained. As shown in FIGS. 7 and 8, the two protruding pieces 34 are not provided to the substantially cylindrical node ring 31c, and two coupling opening portions 312 through which the joint portion 410 is coupled with the bending portion 30 including the node ring 35c are provided to this node ring 31c. The opening portion 312 is a through opening that is formed from the outer peripheral surface 311 toward an inner peripheral surface 310. Moreover, these opening portions 312 are arranged to be apart from each other at substantially 180° in the circumferential direction. Additionally, it is preferable for the opening portions 312 and the protruding pieces 33 to be arranged at positions apart from each other at substantially 90° in the circumferential direction. It is to be noted that the opening portions 312 and the two protruding pieces 33 may be arranged on the same straight line. As a shape of the opening portion 312, a rectangular shape is preferable, hut it does not have to be restricted. Further, the number of the opening portions 312 does not have to be restricted to two.

As shown in FIG. 9, the joint portion 410 arranged at the distal end position of the flexible tube portion 4 is inserted into the node ring 31c. This joint portion 410 is formed of, e.g., a resin and has a hollow shape (e.g., a substantially annular shape). Incidentally, it is good enough for the joint portion 410 to be formed of a material softer than that of the node ring 31c. Furthermore, an outer diameter of the joint portion 410 is substantially the same as an inner diameter of the node ring 31c. Therefore, when the join portion 410 is inserted into the node ring 31c as shown in FIG. 11, a gap can be prevented from being formed between the node ring 31c and the joint portion 410, and these members are fitted as depicted in FIGS. 10 and 11. The flexible tube portion 4 including the joint portion 410 is, e.g., the corrugated tube, and two protruding (projecting) portions 411 each having substantially the same shape as the opening portion 312 are provided on an outer peripheral surface 409 of this joint portion 410. These protruding portions 411 are arranged to be apart from each other at substantially 180° in the circumferential direction in nearly the same manner as the opening portions 312. The protruding portion 411 has a shape that can be fitted into (set in) the opening portion 312 when the joint portion 410 is inserted into the node ring 31c.

That is, when coupling the bending portion 5 with the flexible tube portion 4, the joint portion 410 is inserted into the node ring 31c, and the protruding portions 411 are fitted into the two opening portions 312, respectively. As explained above, each protruding portion 411 has a shape associated with the opening portion 312, the number of the protruding portions 411 is the same as that of the opening portions 312, and the protruding portions 411 are provided in the same positional relationship as the opening portions 312. When the protruding portions 411 are fitted into the opening portions 312, the bending portion 5 is coupled with the flexible tube portion 4. Additionally, in a height direction, each protruding portion 411 slightly protrudes from the opening portion 312 as shown in FIGS. 9 to 11. In the height direction, the opening portions 312 and the protruding portions 411 fitted into the opening portions 312 function as retaining means. It is to be noted that each protruding portion 411 may have the same height as the opening portion 312 in the height direction. In this manner, the opening portions 312 and the protruding portions 411 prevent the bending tube 30 as the annular coupling member and the joint portion 410 from detaching. That is, when coupling the bending portion 5 with the flexible tube portion 4, the opening portions 312 and the protruding portions 411 form the retaining means for preventing the joint portion 410 from detaching from the bending tube 30 as the annular coupling member.

Incidentally, it is preferable for a length of each node ring 31 and a length of the joint portion 410 in the insertion direction of the endoscope 1 to be short. As a result, a hard portion that does not sufficiently bend can be shortened in the coupling portion of the bending portion 5 and the flexible tube portion 4.

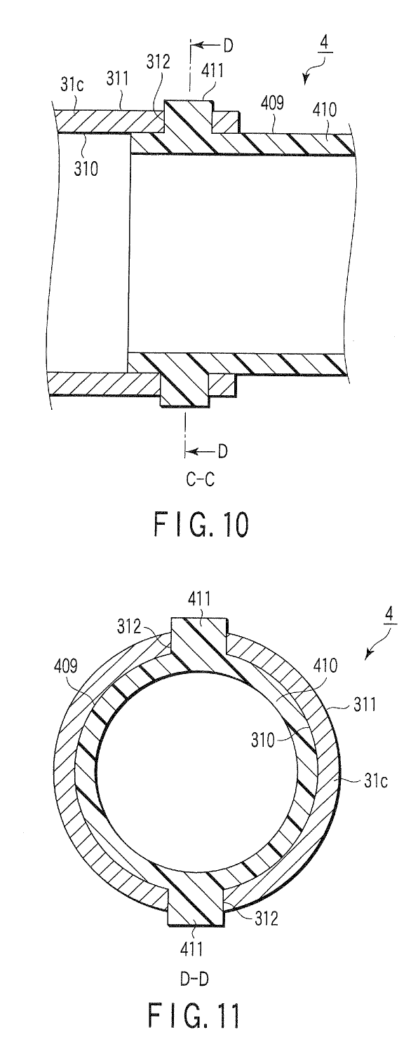

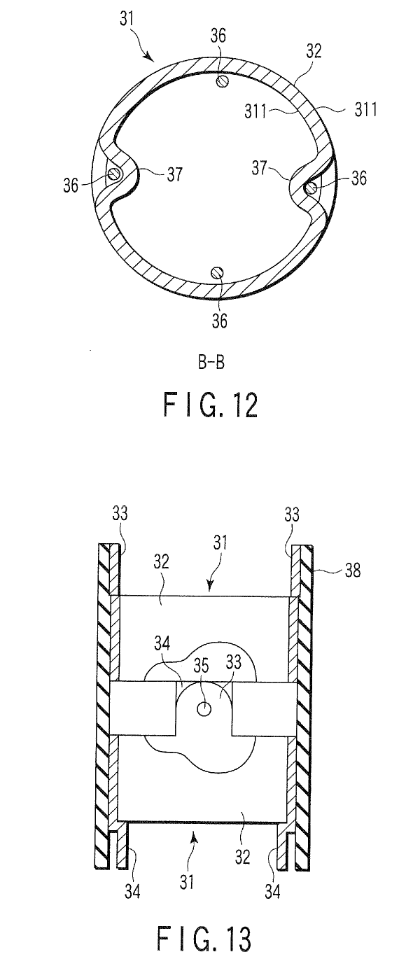

As shown in FIGS. 4 and 12, four operation wires (bending wires) 36 that bend the entire bending portion 5 in four directions, i.e., up, down, left, and right, are arranged in the bending portion 5. A distal end portion of each of these four operation wires 36 is fixed to the rear end portion of the distal end hard portion 6. In detail, each operation wire 36 is fixed to each concave portion 6b based on, e.g., silver brazing, the concave portion 6b being obtained by cutting and bending a part of a distal end side peripheral wall portion associated with each protruding piece 6a based on press working and inwardly protruding the same. The concave portions 6b are formed at four positions shifted at substantially 90° in the circumferential direction. It is to be noted that the distal end portion of each operation wire 36 may be fixed to a concave portion (not shown) formed in the node ring 31a.

Further, two wire guides (wire receivers) 37 are formed on an inner peripheral wall portion of the node ring main body 32 to face the inside as shown in FIGS. 4, 6, and 12. Each wire guide 37 is cut and raised by cutting and bending a part of the peripheral wall portion of the node ring main body 32 from the outer peripheral surface 311 side toward the inner peripheral surface 310 side based on press working and protruding the same. Furthermore, either the vertical operation wire 36 or the lateral operation wire 36 is inserted in each of these wire guides 37.

Each proximal end portion of the operation wires 36 is extended to the inside of the operating section 3 from the inside of the bending portion 5 through the flexible tube portion 4. In the operating section 3 are arranged a non-illustrated vertical bending operation mechanism driven by the vertical bending operation knob 20 and a non-illustrated lateral bending operation mechanism driven by the lateral bending operation knob 21. The proximal end portion of the vertical operation wire 36 is coupled with the vertical bending operation mechanism. The proximal end portion of the lateral operation wire 36 is coupled with the lateral bending operation mechanism. Moreover, each operation wire 36 is driven to be pulled with a swiveling operation of the vertical bending operation knob 20 and the lateral bending operation knob 21. As a result, the bending portion 5 is remotely operated to bend from a regular straight linear state (e.g., a non-bent state indicated by the dashed line in FIG. 1) having a bending angle of 0° to a bent state (indicated by, e.g., the solid line or the chain double-dashed line in FIG. 1) that the bending portion 5 is bent at an arbitrary bending angle in the vertical and lateral directions.

It is to be noted that an envelope tube 38 that is made of an elastic material such as a rubber, formed into the same shape (e.g., a hollow shape or a cylindrical shape) as the bending portion 5 or the flexible tube portion 4, and directly fitted on the outer periphery of the bending tube 30 is provided to the bending portion 5 or the flexible tube portion 4 as shown in FIG. 4 or 13. The envelope tube 38 is injection-molded by using an elastic material having a material quality of a thermoplastic elastomer (e.g., a styrene base, an olefin base, or an urethane base). As a result, the entire outer surface of the bending portion 5 is covered with the envelope tube 38. It is to be noted that molding of the thermoplastic elastomer is not restricted to injection molding, and various kinds of molding methods, e.g., casting, extrusion, or blowing may be applied. Moreover, the material is not restricted to the thermoplastic elastomer, and a rubber material may be used.

Coupling between the node ring 31c in the bending tube 30 and the joint portion 410 in the flexible tube portion 4 in this embodiment will now be explained in detail.

The joint portion 410 is inserted into the node ring 31c. As a result, as shown in FIGS. 9 to 11, the protruding portions 411 are fitted into the two opening portions 312, respectively. Therefore, the bending portion 5 is coupled with the flexible tube portion 4. Additionally, since the inner diameter of the node ring 31c is substantially equal to the outer diameter of the joint portion 410, a gap is prevented from being generated between the node ring 31c and the joint portion 410 when the joint portion 410 is inserted into the node ring 31c, and the joint portion 410 is fitted to the node ring 31c. Therefore, the bending portion 5 and the flexible tube portion 4 (the node ring 31c and the joint portion 410) are firmly coupled with each other.

In this embodiment, the bending portion 5 and the flexible tube portion 4 are coupled with each other by inserting the joint portion 410 into the node ring 31c and fitting the protruding portions 411 in the opening portions 312 in this manner. As a result, the process of coupling the bending portion 5 and the flexible tube portion 4 with each other can be simplified in this embodiment. Further, in this embodiment, the bending portion 5 and the flexible tube portion 4 can be easily coupled with each other. Furthermore, in this embodiment, the bending portion 5 and the flexible tube portion 4 can be coupled with each other in a state where high coupling strength is provided in the insertion direction and a rotating (circumferential) direction of the endoscope 1.

Moreover, in this embodiment, since firm coupling is achieved by the opening portions 312 and the protruding portions 411, the flexible tube portion 4 can be prevented from detaching when operating the endoscope 1. Additionally, since the coupling portion in the node ring 31c and the joint portion 410 is short, the hard portion can be shortened in this embodiment. As a result, excellent insertion properties with respect to the inside of a living body can be assured in this embodiment.

Further, since the process can be simplified and firm coupling can be easily achieved in this embodiment, the endoscope can be configured at a low cost.

It is to be noted that this embodiment can be used in not only the medical endoscope 1 for a body cavity and suchlike but also an industrial (technical) endoscope.

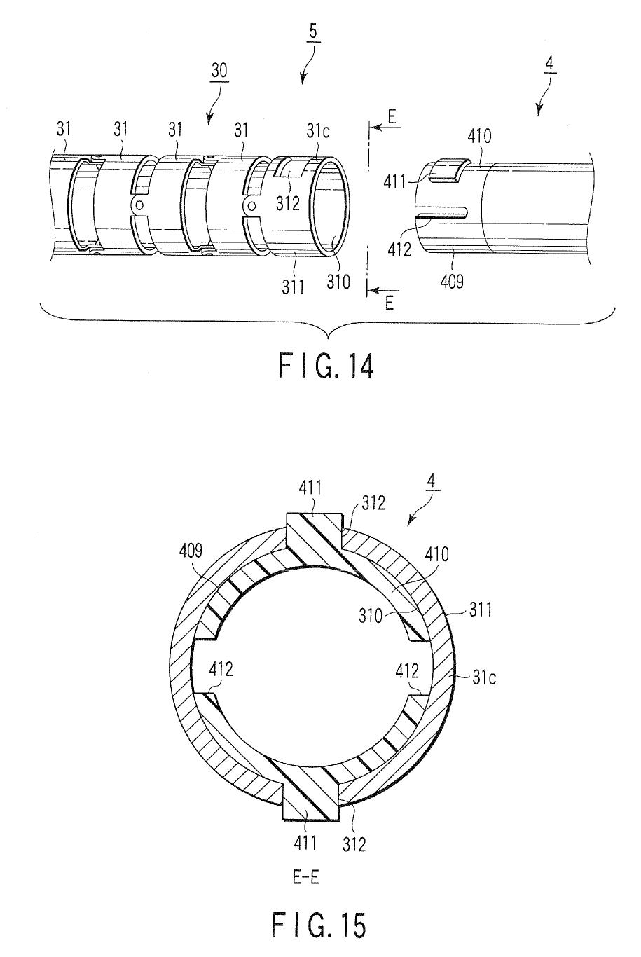

A second embodiment will now be explained with reference to FIGS. 14 and 15.

Like reference numbers denote parts equal to those in the first embodiment to omit detailed explanations of structures, functions, effects, and suchlike thereof. It is to be noted that a structure of a bending portion 5 in this embodiment is substantially equal to that in the first embodiment.

As shown in FIG. 14, a joint portion 410 in this embodiment has two slits 412 as elastic deformation urging portions that bend the joint portion 410 (urge the joint portion 410 to be elastically deformed) to facilitate insertion into a node ring 31c. These slits 412 are arranged to be apart from each other at substantially 180° in a circumferential direction. Furthermore, protruding portions 411 and the slits 412 are arranged to be apart from each other at substantially 90° in the circumferential direction. These slits 412 are linearly formed to extend from a distal end side of the joint portion 410 toward the inside (a proximal end side) along a longitudinal axis direction of the joint portion 410. It is to be noted that the present invention is not restricted to the slits 412 and notches may be provided.

When inserting the joint portion 410 into the node ring 31c, the joint portion 410 is readily bent and easily inserted by the slits 412. At this time, the joint portion 410 is restored to a state before bending by an elastic force of the joint portion 410 formed of a resin and coupled with the node ring 31c without producing a gap between itself and the node ring 31c as shown in FIG. 15 in substantially the same manner as the first embodiment.

As explained above, in this embodiment, providing the slits 412 to the joint portion 410 enables readily bending the joint portion 410 to be inserted into the node ring 31c. It is to be noted that the joint portion 410 is restored to the state before bending by the elastic force of the joint portion 410 formed of a resin in this embodiment, and hence a gap is not produced between the joint portion 410 and the node ring 31c in substantially the same manner as the first embodiment. Additionally, in this embodiment, the protruding portions 411 are fitted into opening portions 312 in substantially the same manner as the first embodiment, thereby coupling a bending portion 5 with a flexible tube portion 4. As a result, the bending portion 5 can be easily coupled with the flexible tube portion 4 in this embodiment, thereby obtaining substantially the same effect as that in the first embodiment.

It is to be noted that the number, arrangement positions, and a shape of the slits 412 in this embodiment do not have to be restricted as explained above, and modifications may be used.

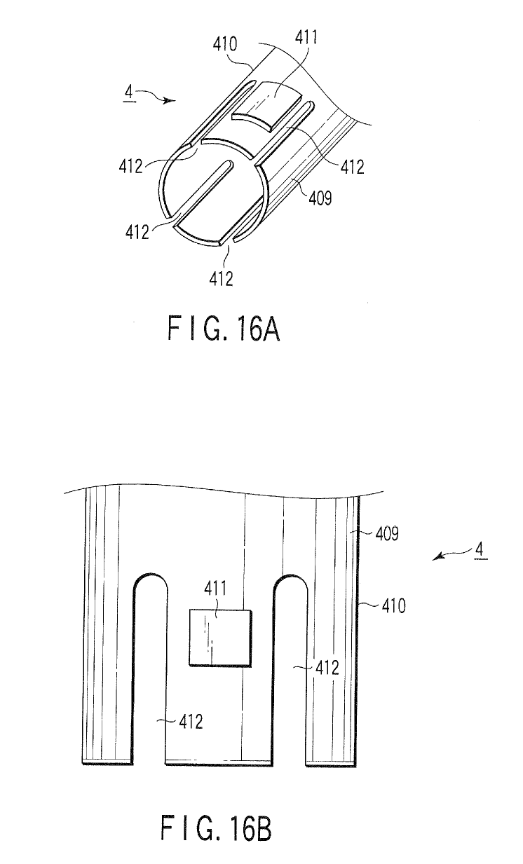

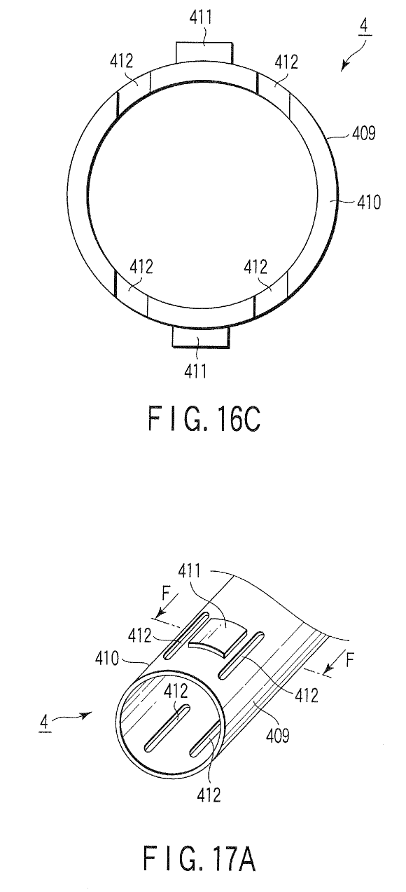

Slits 412 in a first modification may be linearly arranged on both sides of each protruding portion 411 in a longitudinal axis direction of a joint portion 410 as shown in, e.g., FIGS. 16A, 16B, and 16C.

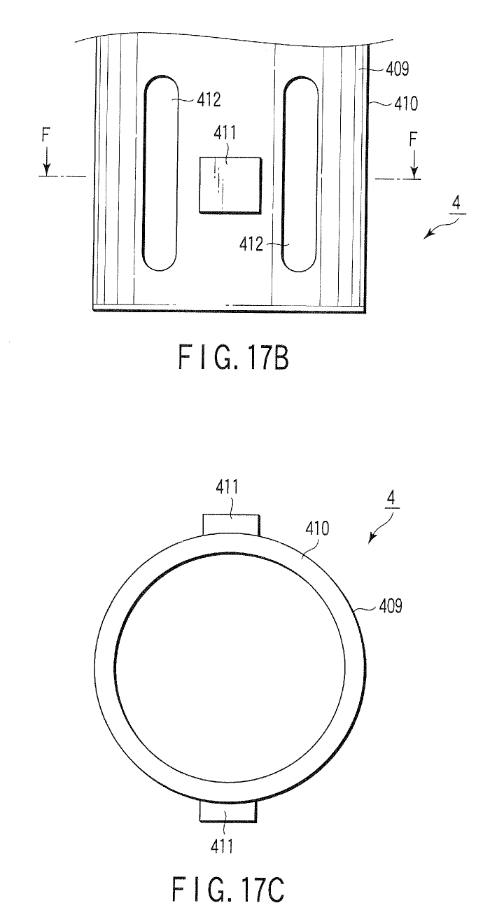

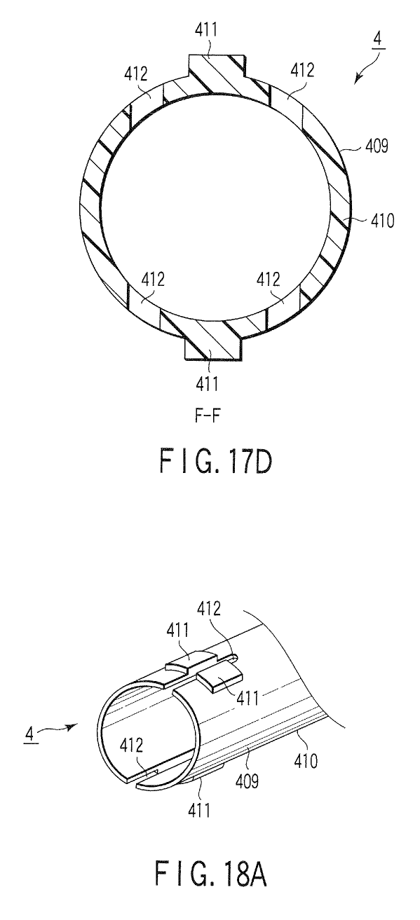

Further, as shown in, e.g., FIGS. 17A, 17B, 17C, and 17D, slits 412 in a second modification may be arranged on both sides of each protruding portion 411 in a longitudinal axis direction of a joint portion 410 in substantially the same manner as the first modification, and they may be formed in the joint portion 410 in the longitudinal axis direction of the joint portion 410

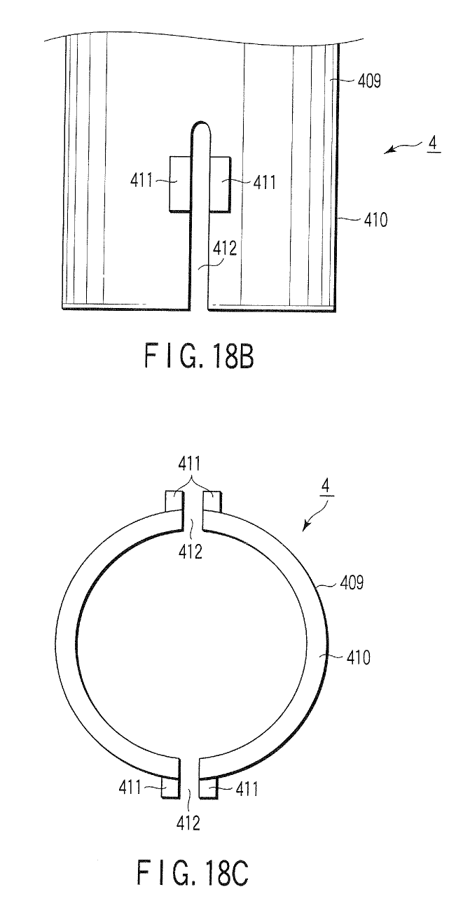

Furthermore, as shown in, e.g., FIGS. 18A, 18B, and 1C, slits 412 in a third modification are formed in a joint portion 410 to be apart from a distal end of the joint portion 410 in a longitudinal axis direction. Moreover, each slit 412 is provided to cut across each protruding portion 411 at a substantially central part of the protruding portion 411 in a circumferential direction (the protruding portions 411 are provided on both sides of each slit 412).

Additionally, as shown in, e.g., FIG. 19, each slit 412 in a fourth modification may have a substantially U-like shape and surround a protruding portion 411. The slit 412 has a slit wide side 412a as one of opposed sides in the substantially U-like shape, a slit wide side 412b as the other of the opposed sides in the substantially U-like shape, and a slid narrow side 412c that is connected with the slit wide side 412a and the slit wide side 412b and has, e.g., an arc-like shape. It is to be noted that the slit narrow side 412c may have a linear shape.

The slit wide sides 412a and 412b are arranged along a longitudinal axis direction of a joint portion 410 (on both sides of the protruding portion 411 in the longitudinal axis direction of the joint portion 410), and the slit narrow side 412c is arranged on a front side of the protruding portion 411 (a node ring 31c side) with respect to an insertion direction of an endoscope 1.

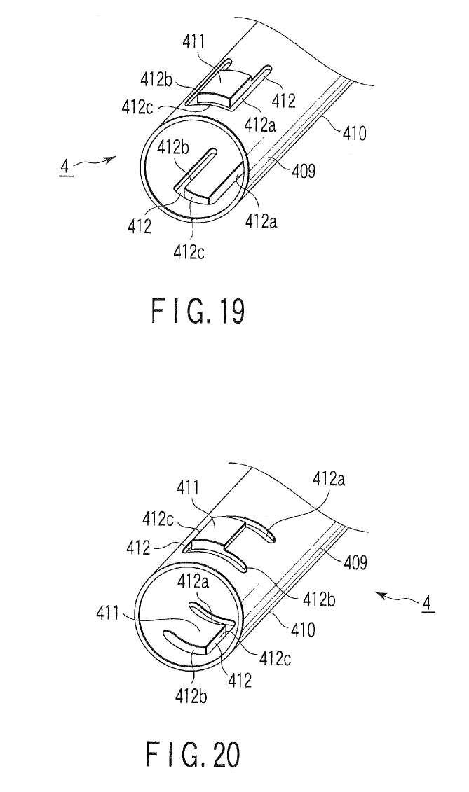

Further, each slit 412 in a fifth modification has a substantially U-like shape like the fourth modification and surrounds a protruding portion 411. It is to be noted that slit wide sides 412a and 412b in this modification are arranged on a front side and a rear side of the protruding portion 411 with respect to an insertion direction of an endoscope 1 and a slit narrow side 412c is arranged at one end of the protruding portion 411 in a longitudinal axis direction of a joint portion 410 as shown in, e.g., FIG. 20. In regard to the slits 412 facing each other in a height direction as shown in FIG. 20, the respective slit narrow sides 412c are not arranged on the same straight line in the height direction. That is, for example, as shown in FIG. 20, the slit narrow side 412c of the slit 412 arranged on the upper side is arranged at a left end of the protruding portion 411 as seen from a node ring 31c, and the slit narrow side 412c of the slit 412 arranged on the lower side is arranged at a right end of the protruding portion 411 as seen from the node ring 31c.

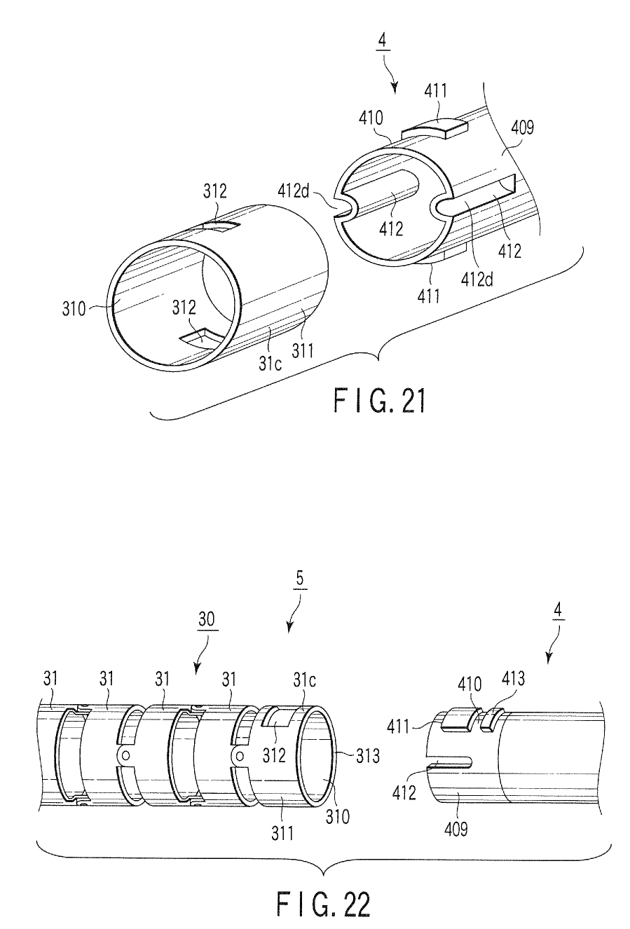

Furthermore, each slit 412 in a sixth modification may have a concave shape 412d obtained by partially concaving a ring of a joint portion 410 in a circumferential direction or a non-illustrated substantially C-like shape as shown in, e.g., FIG. 21. The concave portions 412d are arranged in substantially the same manner as the slits 412 depicted in FIG. 14, and arranged to be exposed on an outer peripheral surface 409. It is to be noted that a shape of each slit 412 does not have to be restricted.

As explained above, the number, a shape, and arrangement positions of the slits 412 in this embodiment do not have to be restricted and the slits 412 can be appropriately provided as desired. Moreover, in this embodiment, above-explained modifications may be combined to provide the slits 412.

A third embodiment will now be explained with reference to FIGS. 22 to 27.

Like reference numbers denote parts equal to those in the foregoing embodiments to omit detailed explanations of structures, functions, effects, and suchlike thereof. It is to be noted that a structure of a bending portion 5 in this embodiment is substantially equal to that in the first embodiment, and a joint portion 410 has slits 412 in substantially the same manner as the second embodiment.

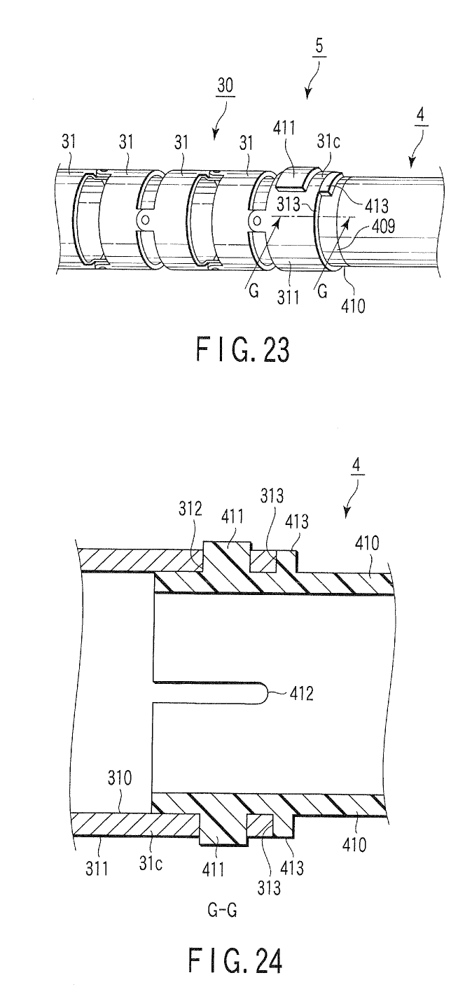

The joint portion 410 in this embodiment has each anti-slip portion 413 that prevents the joint portion 410 from being displaced with respect to a node ring 31c in a thrust (axis) direction when the bending portion 5 and a flexible tube portion 4 (a node ring 35c and the joint portion 41) are coupled with each other as shown in FIG. 22. This anti-slip portion 413 is a protruding portion and arranged behind a protruding portion 411 (a proximal end side of the flexible tube portion 4) with respect to an insertion direction of an endoscope 1. As shown in FIGS. 23 and 24, when the joint portion 410 is inserted into the node ring 31c and the protruding portions 411 are fitted into opening portions 312, the anti-slip portions 413 come into contact with a wall thickness portion (a facet) 313 of the node ring 31c. It is to be noted that a length of each anti-slip portion 413 in a circumferential direction is substantially equal to a length of the protruding portion 411 in the circumferential direction.

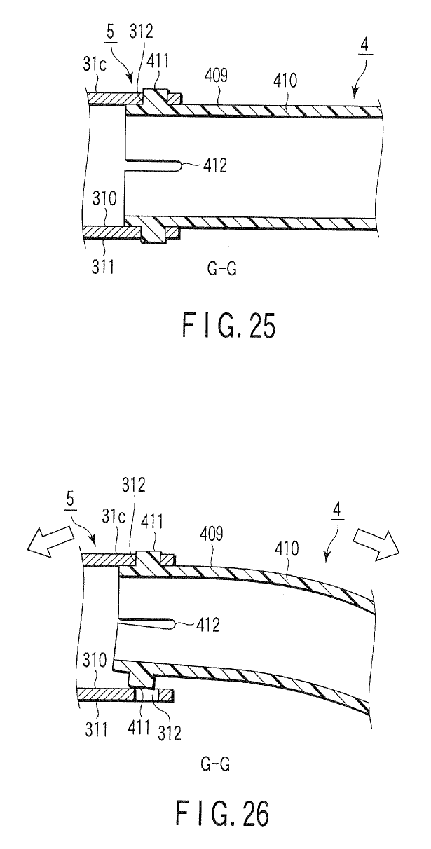

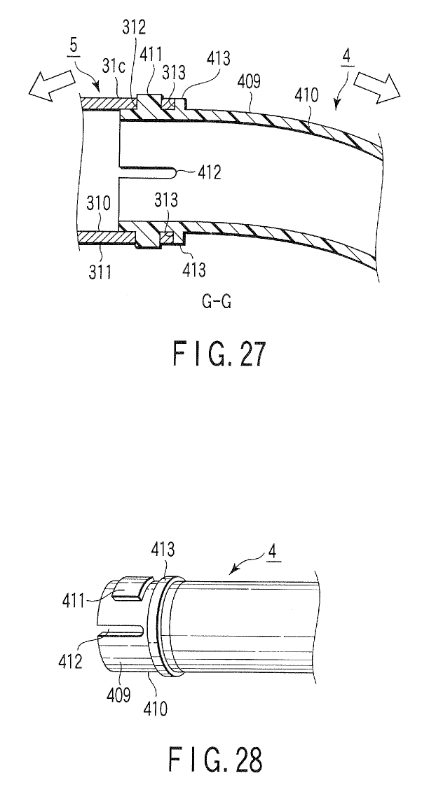

FIG. 25 shows a cross-sectional view of a state where slits 412 are provided in a joint portion 410, the joint portion 410 having no anti-slip portion 413 is inserted into a node ring 31c, protruding portions 411 are fitted into opening portions 312, and a bending portion 5 is coupled with a flexible tube portion 4 taken along line G-G depicted in FIG. 23. The anti-slip portions 413 are not provided. In this state, when a force is applied in a bending direction with respect to the bending portion 5 and the flexible tube portion 4, the joint portion 410 is bent by the slits 412, and the protruding portions 411 may possibly fall off the opening portions 312 as shown in FIG. 26. However, since the anti-slip portions 413 are in contact with the wall thickness portion 313 in this embodiment, the joint portion 410 is not bent when the force is applied in the bending direction, and the anti-slip portions 413 prevent the protruding portions 411 from detaching from the opening portions 312 as shown in FIG. 27.

As explained above, in this embodiment, the anti-slip portions 413 are provided on the outer peripheral surface 409 in the joint portion 410 having the slits 412 provided therein, and the anti-slip portions 413 are brought into contact with the wall thickness portion 313 when the joint portion 410 is inserted into the node ring 31. As a result, this embodiment can obtain substantially the same effect as those in the first embodiment and the second embodiment. Furthermore, in this embodiment, the protruding portions 411 can be prevented from detaching from the opening portions 312 even if a force is applied in the bending direction, thereby further firmly coupling the bending portion 5 and the flexible tube portion 4 with each other.

Incidentally, in this embodiment, it is good enough for at least one of the node ring 35c and the joint portion 410 to have the anti-slip portions 413.

Moreover, a shape or an arrangement position of each anti-slip portion 413 in this embodiment does not have to be restricted, and modifications may be used.

As shown in, e.g., FIG. 28, an anti-slip portion 413 in a first modification is a convex portion that is arranged on an entire outer peripheral surface 409 behind each protruding portion 411 (a proximal end side of a flexible tube portion 4) with respect to an insertion direction of an endoscope 1 along a circumferential direction of a joint portion 410 and comes into contact with a wall thickness portion 313 as a facet of a node ring 35c.

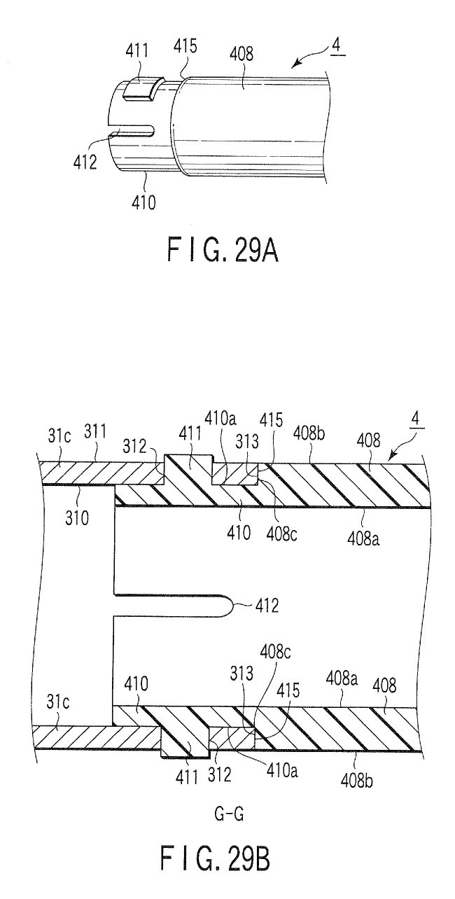

Further, an outer diameter 410a of a joint portion 410 in a second modification is substantially equal to an intermediate diameter 408c as depicted in FIGS. 29A and 29B. The intermediate diameter 408c corresponds to the middle of an inner diameter 408a and an outer diameter 408b of a main body portion 408 of the flexible tube portion 4 arranged behind the joint portion 410. Therefore, a step portion 415 coming into contact with a wall thickness portion 313 is formed between the joint portion 410 and the main body portion 408 as shown in, e.g., FIGS. 29A and 29B. This step portion 415 has an anti-slip function of preventing the joint portion 410 from being displaced with respect to a node ring 31c in a thrust direction when coming into contact with the wall thickness portion 313 of the node ring 31c in substantially the same manner as the anti-slip portion 413. That is, the step portion 415 is an anti-slip portion formed between the joint portion 410 and the main body portion 408.

As explained above, in this modification, the anti-slip portion 413 does not have to be additionally provided in the join portion 410, thereby simplifying a shape of the flexible tube portion 4. Furthermore, when a strong force is applied with respect to a bending portion 5 and the flexible tube portion 4 in a bending direction, the anti-slip portion 413 may be possibly broken from the joint portion as shown in, e.g., FIGS. 27 and 28. However, in this modification, the step portion 415 is prevented from being broken since it is a part of the flexible tube portion 4. Therefore, in this modification, the protruding portions 411 can be prevented from detaching from the opening portions 312 even if a stronger force is applied to the bending portion 5 and the flexible tube portion 4 in the bending direction, thus firmly coupling the bending portion 5 and the flexible tube portion 4 with each other.

Moreover, in this modification, since the step portion 415 is a part of the flexible tube portion 4, the protruding portions 411 can be prevented from detaching from the opening portions 312 at a low cost by simple machining, thus coupling the bending portion 5 with the flexible tube portion 4. Additionally, since a length of the joint portion 410 in the longitudinal axis direction is shortened and the step portion 415 is arranged on the front side with respect to the insertion direction of the endoscope 1, a hard portion that does not sufficiently bend can be shortened in the coupling portion of the bending portion 5 and the flexible tube portion 4.

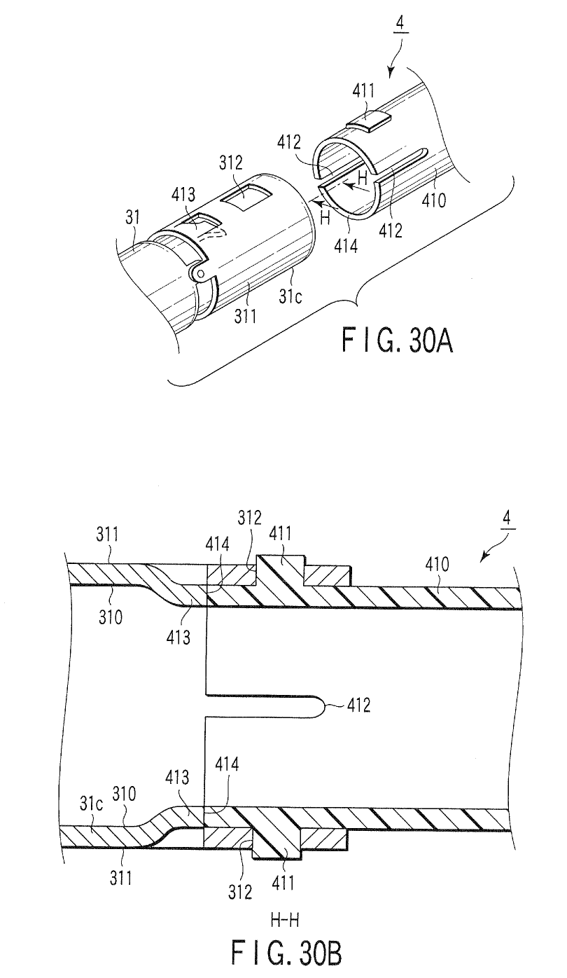

As explained above, although the anti-slip portion 413 or the step portion 415 is provided to the flexible tube portion 4 (the joint portion 410), such a member may be provided to the node ring 31c as shown in, e.g., FIGS. 30A and 30B. An anti-slip portion 413 in this third modification is a cut-and-bent portion that is provided on a front side of each opening portion 312 with respect to an insertion direction of an endoscope 1 and formed to protrude toward the inside of a node ring 31c from an outer peripheral surface 311 of the node ring 31c by cutting and bending based on, e.g., press working. In this case, the anti-slip portion 413 is formed by being cut and bent toward a joint portion 410 along the insertion direction of the endoscope 1.

When the joint portion 410 is inserted into the node ring 31c, a wall thickness portion (a facet) 414 of the joint portion 410 comes into contact with each anti-slip portion 413 as depicted in FIG. 30B.

As a result, in this modification, even if a force is applied in a bending direction in substantially the same manner as each of the foregoing modifications, protruding portions 411 can be prevented from detaching from the opening portions 312, thereby firmly coupling the bending portion 5 with the flexible tube portion 4.

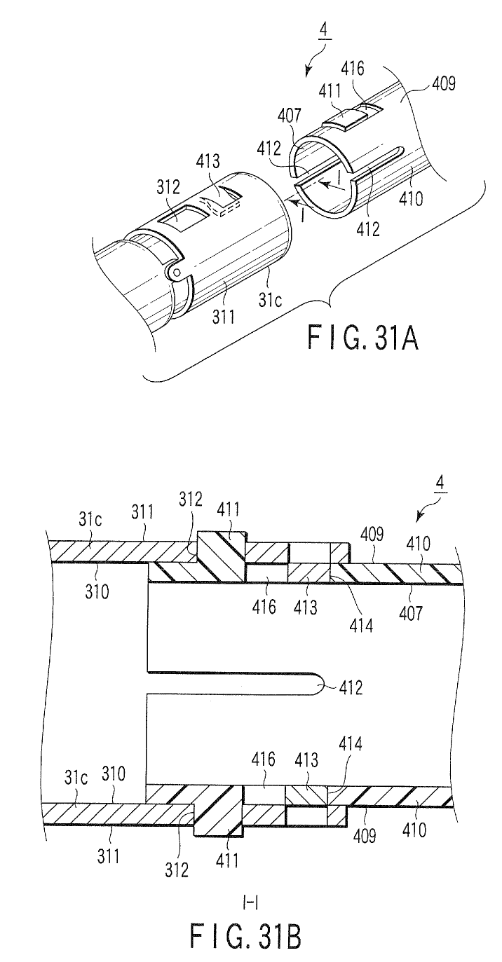

Further, as shown in, e.g., FIGS. 31A and 31B, each anti-slip portion 413 in a fourth modification is a cut-and-bent portion that is provided behind (rear side) each opening portion 312 with respect to an insertion direction of an endoscope 1 and formed to protrude toward the inside of a node ring 31c from an outer peripheral surface 311 of the node ring 31c by cutting and bending based on, e.g., press working. This anti-slip portion 413 is formed along a circumferential direction of the node ring 31c. Furthermore, a joint portion 410 has an opening portion 416 behind a protruding portion 411 in the insertion direction of the endoscope 1. The opening portion 416 is an engagement portion that engages with the anti-slip portion 413. Incidentally, it is preferable for the opening portion 416 to be a through opening that is opened from an outer peripheral surface 409 toward an inner peripheral surface 407 as depicted in FIGS. 31A and 31B.

When the joint portion 410 is inserted into the node ring 31c as shown in FIG. 31B, each anti-slip portion 413 comes into contact with a wall thickness portion (a facet) 414 through the opening portion 416.

As a result, according to this modification, since each anti-slip portion 413 is in contact with the wall thickness portion 414 as explained above, the protruding portions 411 can be prevented from detaching from the opening portions 312 even if a force is applied in the bending direction in substantially the same manner as each of the foregoing modifications, thus further firmly coupling the bending portion 5 and the flexible tube portion 4 with each other. It is to be noted that, in this modification, each anti-slip portion 413 can be provided on the front side of each opening portion 312 in the insertion direction of the endoscope 1 by providing each opening portion 416 on the front side of the protruding portion 411 in the insertion direction of the endoscope 1.

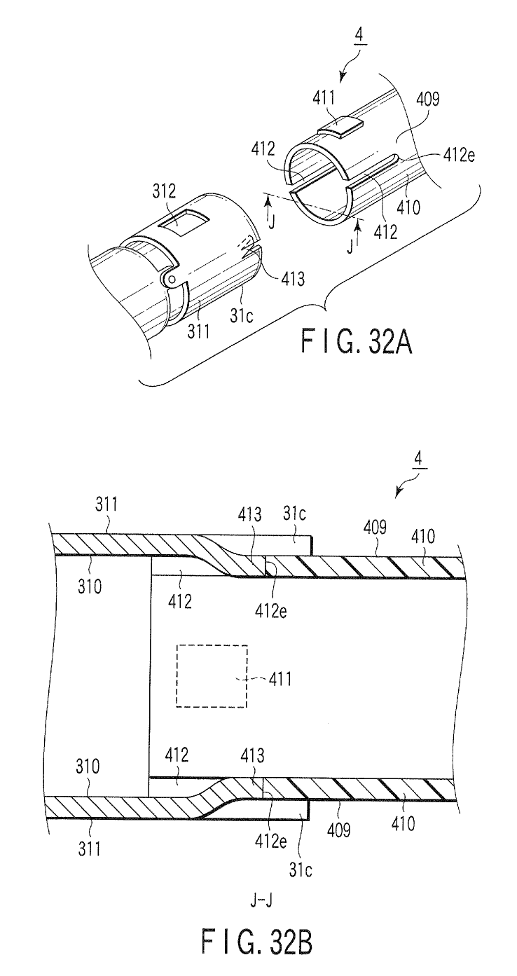

Additionally, an anti-slip portion 413 in a fifth modification is apart from an opening portion 312 at substantially 90° in a circumferential direction and provided at a rim portion on a proximal end side of a node ring 31c arranged behind the opening portion 312 in an insertion direction of an endoscope 1 as shown in, e.g., 32A and 32B. It is to be noted that the anti-slip portion 413 is a cut-and-bent portion formed to protrude toward the inside of the node ring 31c from an outer peripheral surface 311 by cutting and bending based on, e.g., press working. The anti-slip portion 413 is formed along the insertion direction of the endoscope 1. When a joint portion 410 is inserted into the node ring 31c and each protruding portion 411 is fitted into each opening portion 312 as shown in FIG. 32B, each anti-slip portion 413 comes into contact with an abutting portion 412e as an end portion arranged at an end of each slit 412.

Therefore, according to this modification, each protruding portion 411 can be prevented from detaching from each opening portion 312 even if a force is applied in a bending direction in substantially the same manner as each of the foregoing modification, thereby further firmly coupling the bending portion 5 and the flexible tube portion 4 with each other.

It is to be noted that, in this modification, the anti-slip portion 413 is formed from the node ring 31c by cutting and bending based on, e.g., press working to be brought into contact with the abutting portion 412e. Therefore, a member with which the anti-slip portion 413 comes into contact does not have to be additionally provided to the joint portion 410, thus constituting this modification at a low cost.

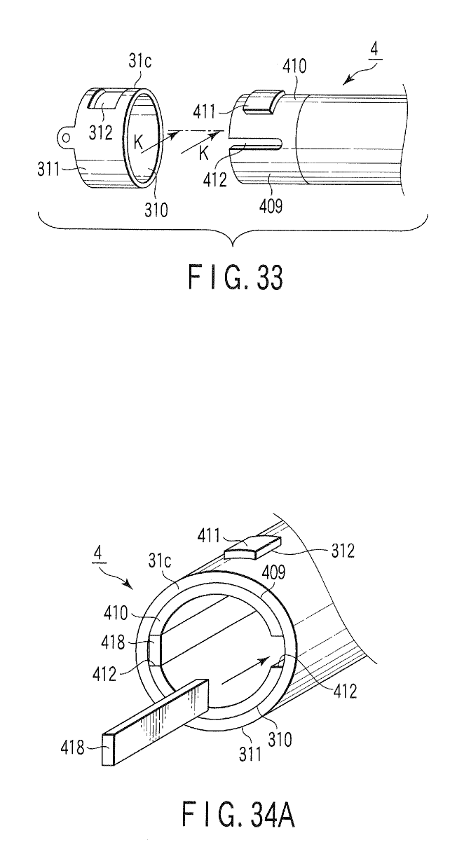

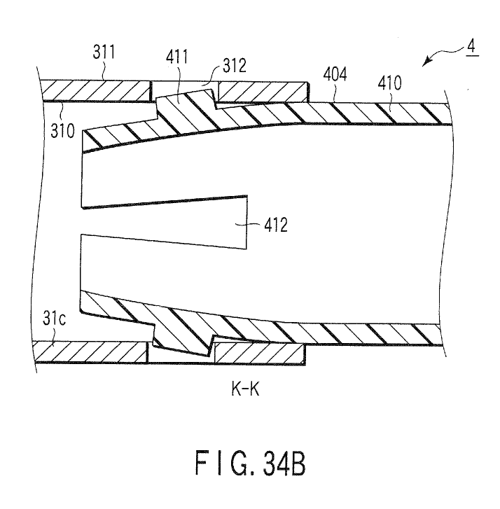

A fourth embodiment will now be explained with reference to FIGS. 33, 34A and 34B.

Like reference numbers denote parts equal to those in each of the foregoing embodiments to omit detailed explanations of structures, function, effects, and suchlike thereof. It is to be noted that a joint portion 410 in this embodiment has slits 412 in substantially the same manner as the second embodiment. Furthermore, in this embodiment, the joint portion 410 is inserted into a node ring 31c before the node ring 31c is coupled with a neighboring node ring 31 as shown in FIG. 34A.

A slit insertion member 418, e.g., a splint having a rectangular solid shape is inserted into each slit 412. When a bending portion 5 and a flexible tube portion 4 are coupled (the joint portion 410 is inserted into the node ring 31c) and a force is applied in a bending direction as shown in, e.g., FIG. 34B, this slit insertion member 418 functions as an anti-falling member (retaining member) that prevents the joint portion 410 from being bent by the slit 412 and also prevents each protruding portion 411 from detaching from (coming off) each opening portion 312.

When the joint portion 410 is inserted into the node ring 31c, each slit insertion member 418 is inserted into the slit 412 from a distal end side of the joint portion 410 along a longitudinal axis direction of the joint portion 410 as shown in FIG. 34A. After the slit insertion member 418 is inserted into the slit 412, the node ring 31c is coupled with the neighboring node ring 31. Incidentally, it is preferable for a length of the slit insertion member 418 in the longitudinal axis direction to be substantially equal to a length of the slit 412 in the longitudinal axis direction.

In this manner, according to this embodiment, after the joint portion 410 is inserted into the node ring 31c, each slit insertion member 418 is inserted into each slit 412. Therefore, this embodiment can obtain substantially the same effect as that of the first embodiment, prevent the slits 412 from being bent even if a force is applied in the bending direction, and also prevent each protruding portion 411 from detaching from each opening portion 312, thereby further firmly coupling the bending portion 5 and the flexible tube portion 4 with each other. Moreover, since a hard portion that does not sufficiently bend can be shorted in the coupling portion of the node ring 31c and the flexible tube portion 4 in substantially the same manner as the foregoing embodiment, thus assuring excellent insertion properties with respect to the inside of a living body.

It is to be noted that a bending preventing method for the slits 412 by the slit insertion members 418 or a coupling method for the bending portion 5 and the flexible tube portion 4 does not have to be restricted to that explained above. Modification using the slit insertion members 418 will now be explained. It is to be noted that each modification is different from the fourth embodiment in that each slit insertion member 418 is inserted in a state where node rings 31 including a node ring 31c are coupled with each other to constitute a bending tube 30 and a joint portion 410 is inserted in the node ring 31c.

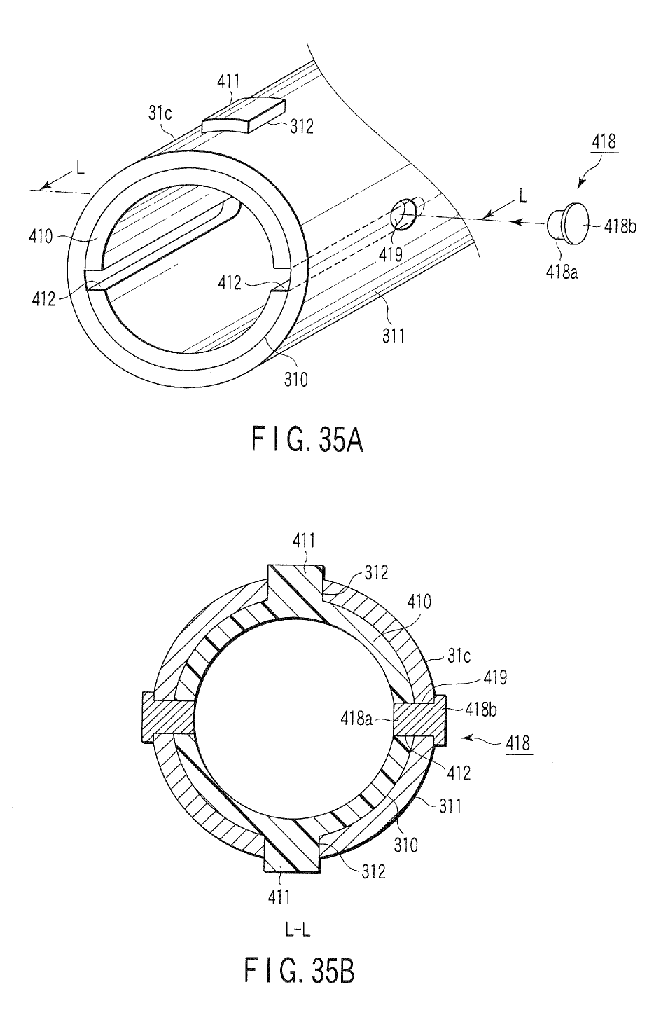

A slit insertion member 418 in a first modification is inserted into a slit 412 via a through-hole 419 provided in a node ring 31c as shown in FIGS. 35A and 35B. In this modification, the slit insertion member 418 is a pin such as a screw having a small-diameter portion 418a and a large-diameter portion 418b having a larger diameter than that of the small-diameter portion 418a. A diameter of the small-diameter portion 418a is substantially equal to a diameter of the through-hole 419 and a width of the slit 412, and the small-diameter portion 418a is fitted into the through-hole 419 and the slit 412. The small-diameter portion 418a reaches the slit 412 via the through-hole 419. A diameter of the large-diameter portion 418b is larger than the diameter of the through-hole 419. Therefore, the large-diameter portion 418b protrudes from an outer peripheral surface 311 and prevents the slit insertion member 418 from being inserted into the slit 412.

The node ring 31c has the through-hole 419 that is arranged on the slit 412 when the joint portion 410 is inserted into the node ring 31c and the protruding portion 411 is fitted into the opening portion 312. It is good enough for at least one through-hole 419 to be provided with respect to one slit 412. Therefore, the plurality of through-holes 419 may be provided to the node ring 31c and the plurality of slit insertion members 418 may be inserted.

After the joint portion 410 is inserted into the node ring 31c, the slit insertion member 418 is inserted into the slit 412 via the through-hole 419. As a result, the slit insertion member 418 is fitted into the through-hole 419 and the slit 412, prevents the joint portion 410 from being bent due to the slit 412, and also prevents the protruding portion 411 from detaching from the opening portion 312 even if a force is applied to the flexible tube portion 4 and the bending portion 5 coupled with each other in the bending direction.

As explained above, according to this modification, after the node rings 31 including the node ring 31c are coupled with each other to constitute the bending tube 30 and the flexible tube portion 4 is coupled with the bending tube 30, the slit insertion member 418 is inserted into the slit 412 via the through-hole 419. As a result, this modification can obtain substantially the same effect as that of the third embodiment. Additionally, according to this modification, the node rings 31 can be coupled with each other to constitute the bending tube 30, and then the bending portion 5 and the flexible tube portion 4 can be firmly coupled with each other like the first embodiment. It is to be noted that, in this modification, a force is not produced in a removal direction of the slit insertion member 418, and hence the slit insertion member 418 may be readily bonded by using an adhesive having thermal melting properties like a hot melt.

Further, a slit insertion member 418 in a second modification is formed of, e.g., a metal material and its small-diameter portion 418a is larger than a width of a slit 412. The width of the slit 412 is smaller than a diameter of a through-hole 419, and the diameter of the through-hole 419 is smaller than a large-diameter portion 418b. Furthermore, an arrangement position of the through-hole 419 is substantially equal to that in the first modification.

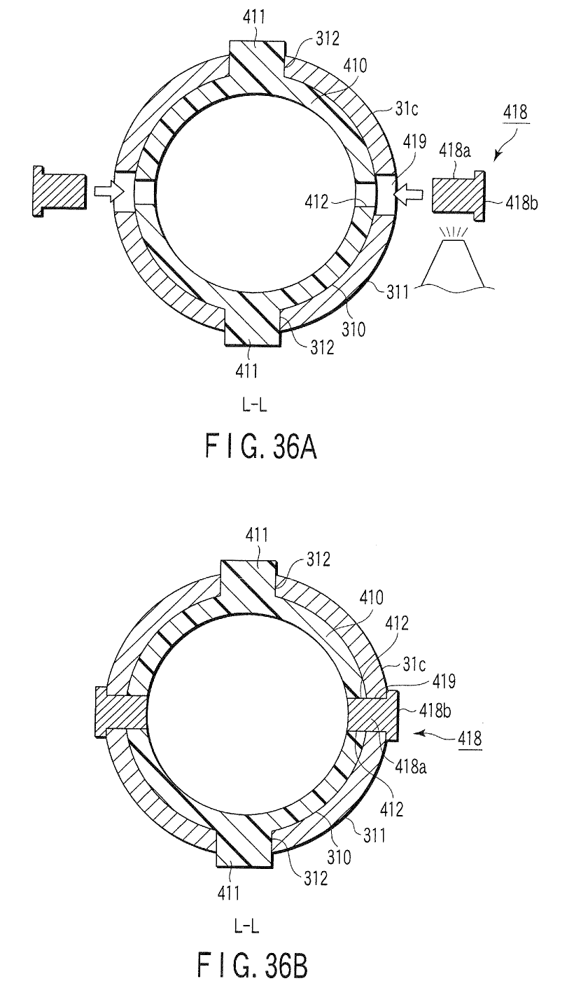

As shown in FIG. 36A, the slit insertion member 418 is heated by, e.g., a torch. The slit insertion member 418 is heated to a temperature at which a resin of a joint portion 410 is softened and melted, and then inserted into the slit 412 via the through-hole 419 as shown in FIG. 36B. At this time, a peripheral wall part of the slit 412 is thermally deformed by the heated small-diameter portion 418, and the small-diameter portion 418a is bonded to a melted part of the melted slit 412.

As explained above, according to this modification, a flexible tube portion 4 is coupled with a bending tube 30, and then the heated slit insertion member 418 is inserted into the slit 412 via the through-hole 419 to bond the slit insertion member 418 to the slit 412. According to this modification, bending of the joint portion 410 due to the slit 412 is avoided by the slit insertion member 418, and each protruding portion 411 can be prevented from detaching from each opening portion 312 even if a force is applied to the flexible tube portion 4 and a bending portion 5 in a bending direction. As a result, this modification can obtain substantially the same effect as that of the third embodiment or the first modification. Further, according to this modification, since the slit insertion member 418 is not subjected to complicated processing, the slit insertion member 418 can be easily bonded to the slit 412 at a low cost. Furthermore, in this modification, bending of the joint portion 410 due to the slit 412 can be avoided, thereby firmly coupling the bending portion 5 and the flexible tube portion 4 with each other.

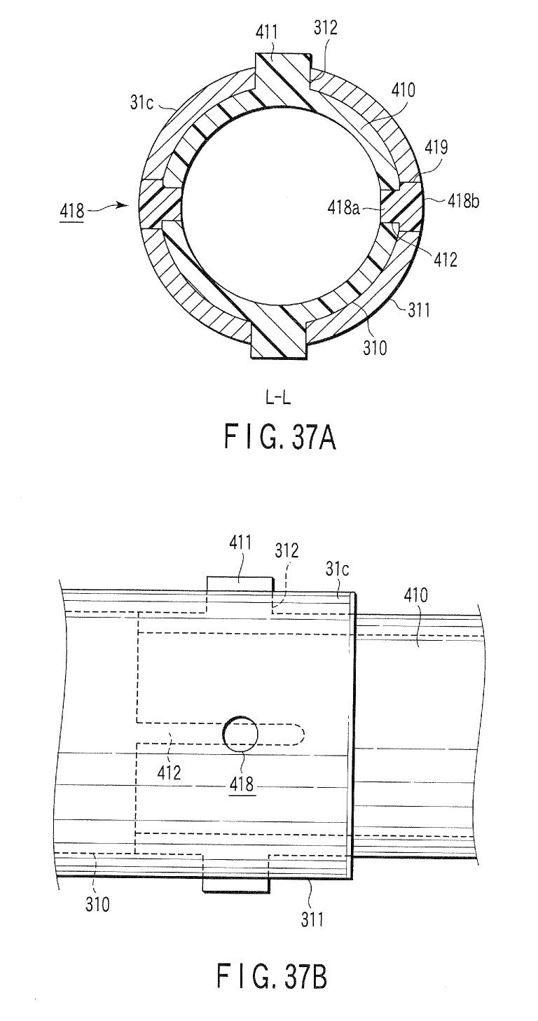

Moreover, a slit insertion member 418 in a third modification is formed of a resin material having, e.g., properties of allowing a laser beam to transmit therethrough, and a joint portion 410 is formed of a resin material having properties of absorbing a laser beam. As shown in FIG. 37A, a width of a slit 412 in this modification is substantially equal to a diameter of a small-diameter portion 418a, and a diameter of a through-hole 419 is substantially equal to a diameter of a large-diameter portion 418b (the width of the slit 412 is smaller than the diameter of the through-hole 419). Therefore, the small-diameter portion 418a is fitted to the width of the slit 412, the large-diameter portion 418b is fitted into the through-hole 419, and the larger-diameter portion 418b does not protrude from an outer peripheral surface 311 of a node ring 31c so that the large-diameter portion 418b is level with the outer peripheral surface 311 as shown in FIG. 37A.

When the slit insertion member 418 is inserted into the slit 412 via the through-hole 419, the small-diameter portion 418a is arranged in the slit 412 and comes into contact with a joint portion 410, and the large-diameter portion 418b comes into contact with the joint portion 410. In this state, when the slit insertion member 418 is irradiated with a laser beam, the laser beam transmitted through the slit insertion member 418 as a laser transmitting material reaches the joint portion 410 on a contact surface of the small-diameter portion 418a and the joint portion 410 and a contact surface of the large-diameter portion 418b and the joint portion 410. The joint portion 410 having properties of absorbing a laser beam absorbs the laser beam, generates heat, and is melted. Then, the contact surfaces of the small-diameter portion 418a and the large-diameter portion 418b that are in contact with the joint portion 410 are melted due to heat produced from the joint portion 410 heated by the laser beam, and a contact surface of the joint portion 410 and the slit insertion member 418 is melted. As explained above, the joint portion 410 and the slit insertion member 418 are melted by the laser beam and bonded to each other.

In this state, the slit insertion member 418 avoids bending of the joint portion 410 due to the slit 412. Moreover, since the slit insertion member 418 is bonded to the joint portion 410, each protruding portion 411 can be prevented from detaching from each opening portion 312 even if a force is applied to the joint portion 410 and a node ring 31c coupled with each other in a bending direction.

As a result, this modification can obtain substantially the same effect as that of the third embodiment or the first and second modifications. Additionally, according to this modification, since the slit insertion member 418 is locally heated, the slit insertion member 418 can be easily bonded to the joint portion 410 by using a laser beam without giving the joint portion 410 an influence of thermal deformation due to the laser beam, for example. Therefore, bending of the joint portion 410 due to the slit 412 can be avoided, and the bending portion 5 and the flexible tube portion 4 can be firmly coupled with each other.

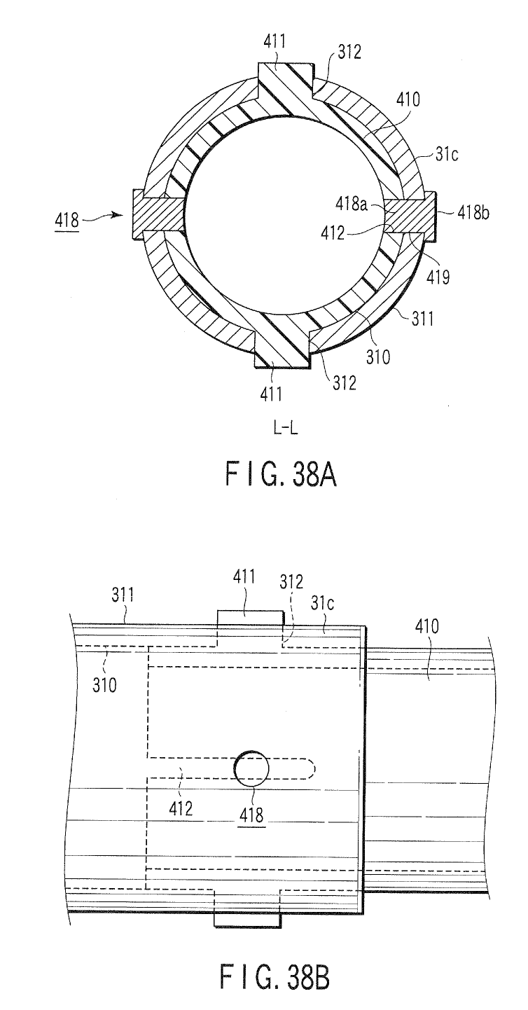

Further, a slit insertion member 418 in a fourth modification is formed of, e.g., a metal material, and a diameter of a through-hole 419 and a width of a slit 412 are substantially equal to a diameter of a small-diameter portion 418a in substantially the same manner as the first modification. Therefore, a large-diameter portion 418b protrudes from an outer peripheral surface 311 as depicted in FIG. 38A.

When the slit insertion member 418 is inserted into the slit 412 via the through-hole 419, the small-diameter portion 418a comes into contact with the slit 412, and the small-diameter portion 418a and the large-diameter portion 418b come into contact with a node ring 31c. In this state, the small-diameter portion 418a, the large-diameter portion 418b, and the node ring 31c are irradiated with a laser beam. On a contact surface of the small-diameter portion 418a and the node ring 31c and a contact surface of the large-diameter portion 418b and the node ring 31c, the small-diameter portion 418a and the node ring 31c are melted (welded) by the laser beam, and the large-diameter portion 418b and the node ring 31c are also melted by the laser beam. As a result, the slit insertion member 418 is welded to the node ring 31c. Therefore, the slit insertion member 418 avoids bending of a joint portion 410 due to the slit 412. Further, since the slit insertion member 418 has adhered to the joint portion 410, each protruding portion 411 is prevented from detaching from each opening portion 312 even if a force is applied to the joint portion 410 and the node ring 31c coupled with each other in a bending direction.

As a result, this modification can obtain substantially the same effect as that of the third embodiment or the first to fourth modifications.

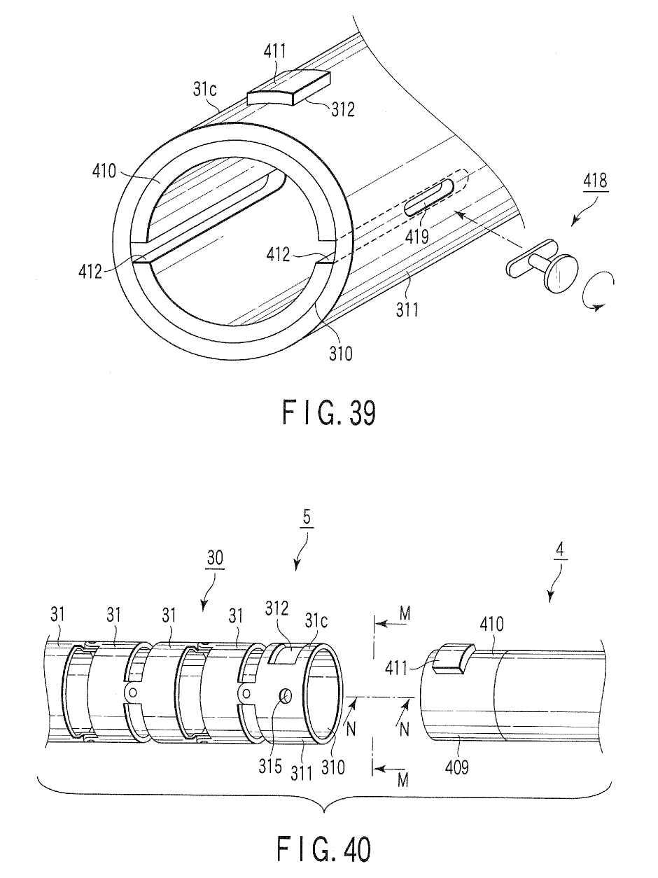

Furthermore, according to a fifth modification, as shown in FIG. 39, a bayonet fastening structure is used to avoid bending of a joint portion 410, and a protruding portion 411 is prevented from detaching from an opening portion 312 even if a force is applied to a flexible tube portion 4 and a bending portion 5 coupled with each other in a bending direction. In detail, a slit insertion member 418 having a structure preferable for the bayonet fastening structure, e.g., a rod-like shape or a cylindrical shape is inserted into a though hole 419 and twisted. As a result, the slit insertion member 418 avoids bending of the joint portion 410 and prevents the protruding portion 411 from detaching from the opening portion 312 even if a force is applied to the flexible tube portion 4 and the bending portion 5 coupled with each other in the bending direction.

Consequently, this modification can obtain substantially the same effect as that of the third embodiment or the first modification.

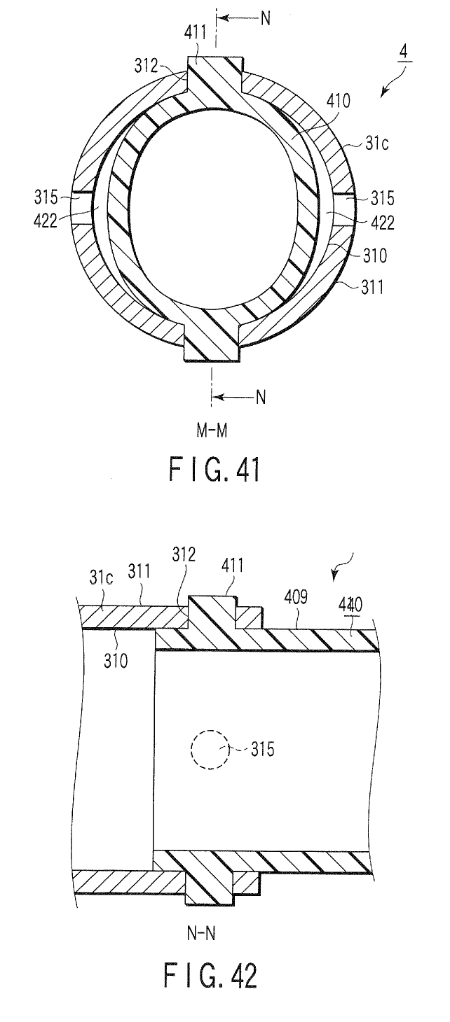

A fifth embodiment will now be explained with reference to FIGS. 40 to 44.

Like reference numbers denote parts equal to those in each of the foregoing embodiments to omit detailed explanations of structures, functions, effects, and suchlike thereof. It is to be noted that a structure of a flexible tube portion 4 is substantially the same as that in the first embodiment.

A joint portion 410 in this embodiment has, e.g., a substantially elliptic shape and has protruding portions 411 on an extension of a major axis as depicted in FIG. 41. A length of the joint portion 410 including a wall thickness length in a major axis direction is substantially equal to an inner diameter of a node ring 31c, and a length of the same in a minor axis direction is shorter than the inner diameter of the node ring 31c. Therefore, when the joint portion 410 is inserted into the node ring 31c as depicted in FIG. 41, the joint portion 410 has a space portion 422 formed between itself and the node ring 31c (in the minor axis direction). The joint portion 410 is bent in the minor axis direction (bent toward the space portion 422) to be readily inserted into the node ring 31c, thereby fitting each protruding portion 411 into each opening portion 312.

The node ring 31c has two insertion holes 315 from which later-explained anti-bending members 424 are inserted into the space portion 422 on an extension of the minor axis (the space portion 422) as shown in FIG. 41. These insertion holes 315 are arranged to be apart from each other at substantially 180° in a circumferential direction. Moreover, each opening portion 312 and each insertion hole 315 are arranged to be apart from each other at substantially 90° in the circumferential direction.

Additionally, the joint portion 410 has the anti-bending members 424 that prevent the joint portion 410 from being bent toward the space portion 422 (in the minor axis direction). The anti-bending members 424 are inserted into the space portion 422 via the insertion holes 315. Such an anti-bending member 424 is, e.g., an adhesive 426 having thermal melting properties like a hot melt as depicted in FIG. 44.

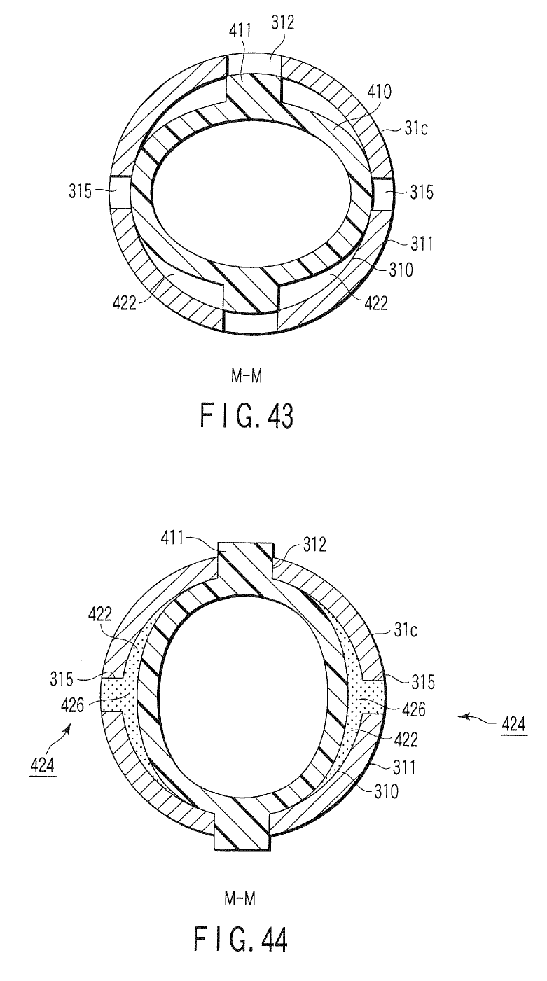

In this embodiment, when a force is applied to the joint portion 410 from both sides in the major axis direction (the protruding portions 411), the joint portion 410 is bent in the minor axis direction as shown in FIG. 43, and it is readily inserted into a node ring 31c. At this time, the joint portion 410 is restored to the state depicted in FIG. 41 by, e.g., an elastic force, and each protruding portion 411 is fitted into each opening portion 312 in substantially the same manner as the first embodiment. In this state, the adhesive 426 is inserted into the space portion 422 from the insertion holes 315 as depicted in FIG. 44, and the space portion 422 is filled with the adhesive 426. As a result, in the joint portion 410, when a force is applied in the bending direction, the adhesive 426 avoids bending of the joint portion 410 in the minor axis direction, thereby preventing each protruding portion 411 from detaching from each opening portion 312. In this manner, the anti-bending members 424 prevents the joint portion 410 from being bent in the minor axis direction (restricts elastic deformation).

In this manner, according to this embodiment, since the joint portion 410 has an elliptic shape, the joint portion 410 can be readily inserted into a node ring 31c when it is bent in the minor axis direction. Furthermore, after the joint portion 410 is inserted into the node ring 31c, inserting each anti-bending member 424 into the space portion 422 enables avoiding bending of the joint portion 410 in the minor axis direction even if a force is applied in the bending direction, thus preventing each protruding portion 411 from detaching from each opening portion 312. Therefore, this embodiment can obtain substantially the same effect as that of the first embodiment and avoid bending of the joint portion 410 in the minor axis direction, thereby further readily coupling the bending portion 5 and the flexible tube portion 4 with each other.

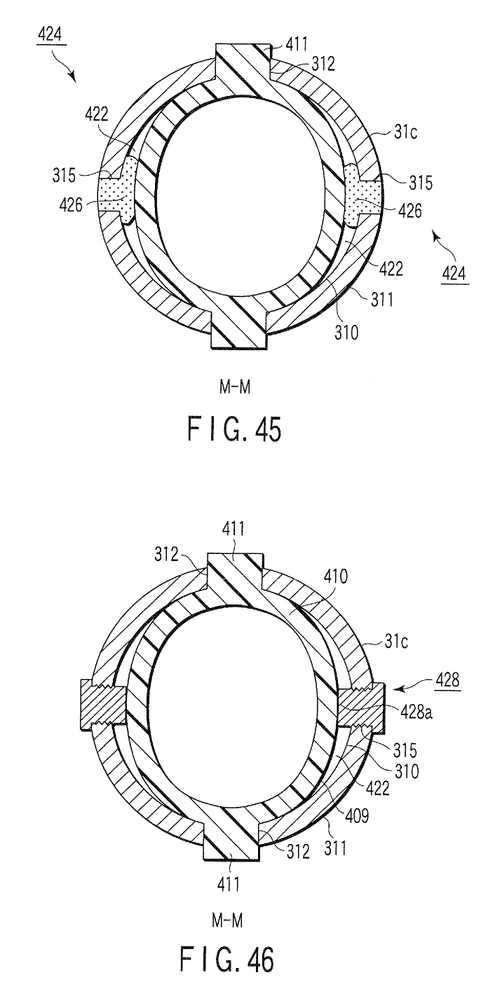

It is to be noted that the adhesive 426 may be inserted into the space portion 422 alone which is arranged between each insertion hole 315 and the joint portion 410 as shown in FIG. 45 as long as the joint portion 410 can be prevented from being bent in the minor axis direction. Moreover, the adhesive 426 intended to avoid bending does not have to be restricted to an adhesive, e.g., a hot melt as long as it is solidified after being injected from each hole, and a silicon rubber or a silicon sealing having low adhesion properties may be used.

Additionally, like the fourth embodiment, when the joint portion 410 is inserted into the node ring 31c before coupling the node ring 31c with a neighboring node ring 31, inserting the adhesive 426 into the space portion 422 from a distal end portion of the joint portion 410 in the major axis direction enables omitting the insertion holes 315.

Further, although the adhesive 426 is used as the anti-bending member 424 that avoids bending of the joint portion 410 in the minor axis direction in this embodiment, the present invention does not have to be restricted thereto. For example, as a first modification, the anti-bending member 424 may be a screw 428 as depicted in FIG. 46. This screw is inserted into a space portion 422 via an insertion hole 315 to be fitted into the insertion hole 315 after a joint portion 410 is inserted into a node ring 31c and each protruding portion 411 is fitted into each opening portion 312. Furthermore, a distal end 428a comes into contact with an outer peripheral surface 409. As a result, the screw 428 can avoid bending of the joint portion 410 in a minor axis direction even if a force is applied in a bending direction, and this modification can obtain substantially the same effect as that of the fifth embodiment.

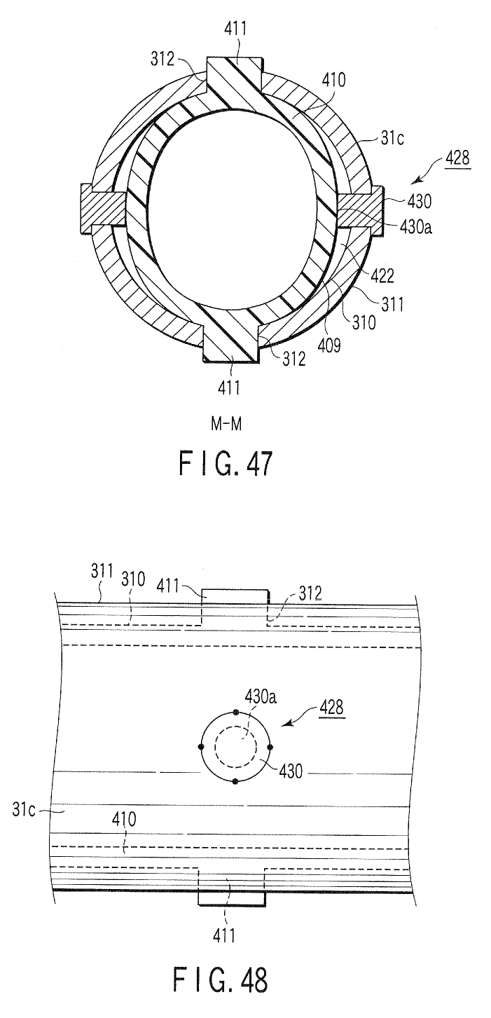

Moreover, as a second modification, the anti-bending member 424 may be a metal pin 430 as depicted in FIG. 47. This pin 430 is inserted into a space portion 422 via an insertion hole 315 to be welded to a node ring 31c by a laser beam after a joint portion 410 is inserted into the node ring 31c and a protruding portion 411 is fitted into an opening portion 312. In this modification, it is preferable to perform welding at four positions on a circumference of the pin 430 at equal intervals as shown in FIG. 48. It is to be noted that, when the pin 430 is welded, a distal end 430a comes into contact with an outer peripheral surface 409. As a result, the pin 430 can avoid bending of the joint portion 410 in a minor axis direction even if a force is applied in a bending direction, and this modification can obtain substantially the same effect as that of the fifth embodiment.

As explained above, it is good enough for the anti-bending member 424 to avoid bending of the joint portion 410 in the minor axis direction even if a force is applied in the bending direction.

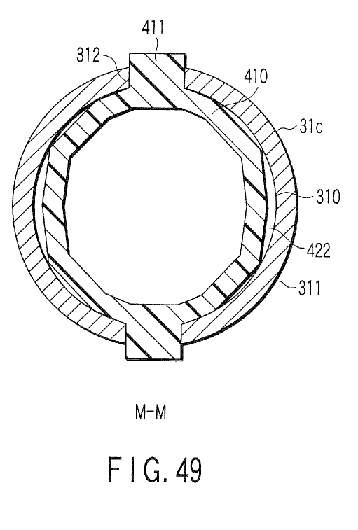

It is to be noted that the joint portion 410 may be formed into a polygonal shape as long as it has a substantially elliptic shape as shown in FIG. 49.

What is claimed is:

1. A coupling structure for an endoscope flexible tube and an annular coupling member, comprising:
a joint portion that is arranged at a distal end portion of a flexible tube portion for an endoscope and has a substantially annular shape;
an annular coupling member that is provided to a bending portion arranged on a distal end side of the flexible tube portion and coupled with the joint portion;
a coupling opening portion that is provided in the annular coupling member and used to couple the joint portion with the annular coupling member; and
a protruding portion that is formed on an outer peripheral surface of the joint portion and configured to be fitted into the opening portion,
wherein, when the protruding portion is fitted into the opening portion at the time of coupling that the joint portion is inserted into the annular coupling member to couple the flexible tube portion with the bending portion, the opening portion and the protruding portion form retaining means for preventing the joint portion coming off the annular coupling member.

2. The coupling structure for an endoscope flexible tube and an annular coupling member according to claim 1, wherein the annular coupling member is formed of a hard material, and the joint portion is formed of a material softer than the annular coupling member.

3. The coupling structure of an endoscope flexible tube and an annular coupling member according to claim 1, wherein the joint portion has an elastic deformation urging portion that facilitates elastic deformation of the joint portion and is linearly formed from a distal end side toward a proximal end side of the joint portion in a longitudinal axis direction of the joint portion.

4. The coupling structure for an endoscope flexible tube and an annular coupling member according to claim 3, wherein the elastic deformation urging portion has a slit.

5. The coupling structure for an endoscope flexible tube and an annular coupling member according to claim 3, wherein the elastic deformation urging portion has a concave portion obtained by concaving a part of a ring of the joint portion in a circumferential direction.

6. The coupling structure for an endoscope flexible tube and an annular coupling member according to claim 3, wherein at least one of the annular coupling member and the joint portion has an anti-slip portion that prevents the joint portion from being displaced from the annular coupling member in an axial direction of the annular coupling member and the joint portion when coupling the bending portion and the flexible tube portion with each other.

7. The coupling structure for an endoscope flexible tube and an annular coupling member according to claim 6, wherein the anti-slip portion is provided to the joint portion and arranged behind the protruding portion in an insertion direction of the endoscope.

8. The coupling structure for an endoscope flexible tube and an annular coupling member according to claim 7, wherein the anti-slip portion is a convex portion that is arranged on an entire outer peripheral surface of the joint portion along the circumferential direction and comes into contact with a facet of the annular coupling member.

9. The coupling structure for an endoscope flexible tube and an annular coupling member according to claim 7, wherein the anti-slip portion is a step portion that is formed between the joint portion and a main body portion of the flexible tube portion arranged behind the joint portion and comes into contact with a facet of the annular coupling member.

10. The coupling structure for an endoscope flexible tube and an annular coupling member according to claim 6, wherein the anti-slip portion provided to the annular coupling member has a cut-and-bent portion that protrudes toward the inside from the outer peripheral surface of the annular coupling member by cutting and bending on a front side of the opening portion with respect to the insertion direction of the endoscope, is formed along the insertion direction of the endoscope, and comes into contact with a facet of the joint portion.

11. The coupling structure for an endoscope flexible tube and an annular coupling member according to claim 6, wherein the anti-slip portion provided to the annular coupling member has a cut-and-bent portion that protrudes toward the inside from the outer peripheral surface of the annular coupling member by cutting and bending on a rear side of the opening portion with respect to the insertion direction of the endoscope and is formed along the circumferential direction of the annular coupling member, and
the joint portion has an engagement portion that engages with the cut-and-bent portion behind the protruding portion.

12. The coupling structure for an endoscope flexible tube and an annular coupling member according to claim 6, wherein the anti-slip portion provided to the annular coupling member has a cut-and-bent portion that protrudes toward the inside from the outer peripheral surface of the annular coupling member by cutting and bending at a rim portion of the annular coupling member on the proximal end side in the insertion direction of the endoscope and comes into contact with an end portion of the elastic deformation urging portion.

13. The coupling structure for an endoscope flexible tube and an annular coupling member according to claim 6, wherein an retaining member that prevents the protruding portion from coming off the opening portion when the joint portion is bent is inserted into the elastic deformation urging portion in a coupled state where the joint portion is inserted into and coupled with the annular coupling member.

14. The coupling structure for an endoscope flexible tube and an annular coupling member according to claim 13, wherein the retaining member is inserted into the elastic deformation urging portion from the distal end side of the joint portion along the longitudinal axis direction of the joint portion.

15. The coupling structure for an endoscope flexible tube and an annular coupling member according to claim 13, wherein the annular coupling member has a through-hole arranged above the elastic deformation urging portion when the joint portion is inserted into the annular coupling member and the protruding portion is fitted into the opening portion, and
the retaining member is inserted into the elastic deformation urging portion via the through-hole in a coupled state where the joint portion is inserted into and coupled with the annular coupling member.

16. The coupling structure for an endoscope flexible tube and an annular coupling member according to claim 15, wherein the retaining member is formed of a metal material, a circumferential wall part of the elastic deformation urging portion is thermally deformed by the heated retaining member when the heated retaining member is inserted into the elastic deformation urging portion via the through-hole, and the retaining member is bonded to a melted part of the melted elastic deformation urging portion.

17. The coupling structure for an endoscope flexible tube and an annular coupling member according to claim 15, wherein the retaining member is formed of a resin material, and the retaining member and the joint portion are melted to adhere to each other by a laser beam when the retaining member is inserted into elastic deformation urging portion via the through-hole.

18. The coupling structure for an endoscope flexible tube and an annular coupling member according to claim 15, wherein the retaining member is formed of a metal material, and the retaining member is welded to the annular coupling member by a laser beam when the retaining member is inserted into the elastic deformation urging portion via the through-hole.

19. The coupling structure for an endoscope flexible tube and an annular coupling member according to claim 1, wherein the joint portion has a space portion formed between itself and the annular coupling member and an anti-bending member that prevents the joint portion from being bent toward the space portion at the time of coupling that the joint portion is inserted into and coupled with the annular coupling member.

20. The coupling structure for an endoscope flexible tube and an annular coupling member according to claim 19, wherein the annular coupling member has an insertion hole through which the anti-bending member is inserted into the space portion.
